(19) United States
(12) Patent Application Publication
(10) Pub. No.: US 2022/0013238 A1
(43) Pub. Date: Jan. 13, 2022

(54) SYSTEMS AND METHODS FOR DETERMINING INDICATORS OF RISK OF PATIENTS TO AVOIDABLE HEALTHCARE EVENTS AND PRESENTATION OF THE SAME

(71) Applicant: UNIVERSITY OF MARYLAND, BALTIMORE COUNTY, Baltimore, MD (US)

(72) Inventors: Ian STOCKWELL, Cooksville, MD (US); Fei HAN, Baltimore, MD (US); Morgan HENDERSON, Baltimore, MD (US)

(21) Appl. No.: 17/359,821

(22) Filed: Jun. 28, 2021

Related U.S. Application Data
(60) Provisional application No. 63/045,418, filed on Jun. 29, 2020.

(51) Int. Cl. G16H 50/30; G16H 50/70; G16H 50/20; G16H 10/60

(52) U.S. Cl. CPC G16H 50/30; G16H 10/60; G16H 50/20; G16H 50/70

(57) ABSTRACT

Systems and methods for determining indicators of risk of patients to avoidable healthcare events and presentation of the same are disclosed. According to an aspect, a method includes receiving, from a database, data associated with a plurality of individuals. The method also includes correlating the data to an avoidable healthcare event for one or more of the individuals. Further, the method includes generating a model that relates the data to the avoidable healthcare event based on the correlation of the data to the avoidable healthcare event. The method also includes applying the model to data of another individual to generate an indicator of risk of the other individual to the avoidable healthcare event. Further, the method includes presenting the indicator of risk via a user interface.

### CROSS REFERENCE TO RELATED APPLICATION

This application claims priority to U.S. Provisional Patent Application No. 63/045,418, filed Jun. 29, 2020, and titled PREDICTING/PREVENTING AVOIDABLE HOSPITAL EVENTS (PRE-AH), the content of which is incorporated herein by reference in its entirety.

### TECHNICAL FIELD

The presently disclosed subject matter relates generally to healthcare services and resources. Particularly, the presently disclosed subject matter relates to systems and methods for determining indicators of risk of patients to avoidable healthcare events and presentation of the same.

### BACKGROUND

With regard to healthcare services, there is a limited number of healthcare facilities and resources available to a growing population needing such services. Many healthcare facilities face problems associated with managing capacity and resources as well as the flow of patients through the healthcare facilities. Also, there will be further demands as the aging population continues to grow. To adequately meet these demands, the capacity of healthcare services must either grow or become more efficient.

The management of healthcare services has become more efficient due to the use of computers to handle patient records, to manage the care of patients, and to manage healthcare resources. As result, the healthcare service capacity has improved. These computer systems must also assure that high quality services are provided while improving efficiency and capacity.

In view of the foregoing, there is a continuing need to improve the efficiency and capacity of healthcare services. Also, there is a continuing need to improve or at least maintain a high level of quality of healthcare services.

### BRIEF DESCRIPTION OF THE DRAWINGS

Having thus described the presently disclosed subject matter in general terms, reference will now be made to the accompanying Drawings, which are not necessarily drawn to scale, and wherein:

FIG. 1 is a block diagram of a system configured to determine and present risk indicators of patients to avoidable healthcare events in accordance with embodiments of the present disclosure;

FIG. 2 is a flow diagram of an example method for determining and presenting risk indicators of patients to avoidable healthcare events in accordance with embodiments of the present disclosure;

FIG. 3 depicts a diagram showing stratification of a population of individuals by practices/physicians;

FIG. 4 is a graph showing test results of a standard neural network model in accordance with embodiments of the present disclosure;

FIG. 5 is a graph showing concentration curves based on avoidable hospital events for a period of time;

FIG. 6 is a graph that plots the Gini coefficients from 23 months of data based on the concentration curves estimated on the 20 percent holdout sample from each month;

FIG. 7 is a flow diagram of an example method for determining indicators of risk of patients to avoidable healthcare events in accordance with embodiments of the present disclosure;

FIG. 8 is a flow diagram of an example method for scoring and file transfer processing in accordance with embodiments of the present disclosure;

FIG. 9 is a diagram showing code modularity according to embodiments of the present disclosure; and

FIG. 10 is a screenshot of a display of a patient's data including likelihood of avoidable hospital event in accordance with embodiments of the present disclosure.

### SUMMARY

The presently disclosed subject matter relates to systems and methods for determining indicators of risk of patients to avoidable healthcare events and presentation of the same. According to an aspect, a method includes receiving, from a database, data associated with a plurality of individuals. The method also includes correlating the data to an avoidable healthcare event for one or more of the individuals. Further, the method includes generating a model that relates the data to the avoidable healthcare event based on the correlation of the data to the avoidable healthcare event. The method also includes applying the model to data of another individual to generate an indicator of risk of the other individual to the avoidable healthcare event. Further, the method includes presenting the indicator of risk via a user interface.

### DETAILED DESCRIPTION

The following detailed description is made with reference to the figures. Exemplary embodiments are described to illustrate the disclosure, not to limit its scope, which is defined by the claims. Those of ordinary skill in the art will recognize a number of equivalent variations in the description that follows.

Articles “a” and “an” are used herein to refer to one or to more than one (i.e. at least one) of the grammatical object of the article. By way of example, “an element” means at least one element and can include more than one element.

“About” is used to provide flexibility to a numerical endpoint by providing that a given value may be “slightly above” or “slightly below” the endpoint without affecting the desired result.

The use herein of the terms “including,” “comprising,” or “having,” and variations thereof is meant to encompass the elements listed thereafter and equivalents thereof as well as additional elements. Embodiments recited as “including,” “comprising,” or “having” certain elements are also contemplated as “consisting essentially of” and “consisting” of those certain elements.

Unless otherwise defined, all technical terms used herein have the same meaning as commonly understood by one of ordinary skill in the art to which this disclosure belongs.

As referred to herein, the terms “computing device” and “entities” should be broadly construed and should be understood to be interchangeable. They may include any type of computing device, for example, a server, a desktop computer, a laptop computer, a smartphone, a cell phone, a pager, a personal digital assistant (PDA, e.g., with GPRS NIC), a mobile computer with a smartphone client, or the like.

As referred to herein, a user interface is generally a system by which users interact with a computing device. A user interface can include an input for allowing users to manipulate a computing device, and can include an output for allowing the system to present information and/or data, indicate the effects of the user's manipulation, etc. An example of a user interface on a computing device (e.g., a mobile device) includes a graphical user interface (GUI) that allows users to interact with programs in more ways than typing. A GUI typically can offer display objects, and visual indicators, as opposed to text-based interfaces, typed command labels or text navigation to represent information and actions available to a user. For example, an interface can be a display window or display object, which is selectable by a user of a mobile device for interaction. A user interface can include an input for allowing users to manipulate a computing device, and can include an output for allowing the computing device to present information and/or data, indicate the effects of the user's manipulation, etc. An example of a user interface on a computing device includes a graphical user interface (GUI) that allows users to interact with programs or applications in more ways than typing. A GUI typically can offer display objects, and visual indicators, as opposed to text-based interfaces, typed command labels or text navigation to represent information and actions available to a user. For example, a user interface can be a display window or display object, which is selectable by a user of a computing device for interaction. The display object can be displayed on a display screen of a computing device and can be selected by and interacted with by a user using the user interface. In an example, the display of the computing device can be a touch screen, which can display the display icon. The user can depress the area of the display screen where the display icon is displayed for selecting the display icon. In another example, the user can use any other suitable user interface of a computing device, such as a keypad, to select the display icon or display object. For example, the user can use a track ball or arrow keys for moving a cursor to highlight and select the display object.

The display object can be displayed on a display screen of a mobile device and can be selected by and interacted with by a user using the interface. In an example, the display of the mobile device can be a touch screen, which can display the display icon. The user can depress the area of the display screen at which the display icon is displayed for selecting the display icon.

In another example, the user can use any other suitable interface of a mobile device, such as a keypad, to select the display icon or display object. For example, the user can use a track ball or times program instructions thereon for causing a processor to carry out aspects of the present disclosure.

As used herein, the term “memory” is generally a storage device of a computing device. Examples include, but are not limited to, read-only memory (ROM) and random access memory (RAM).

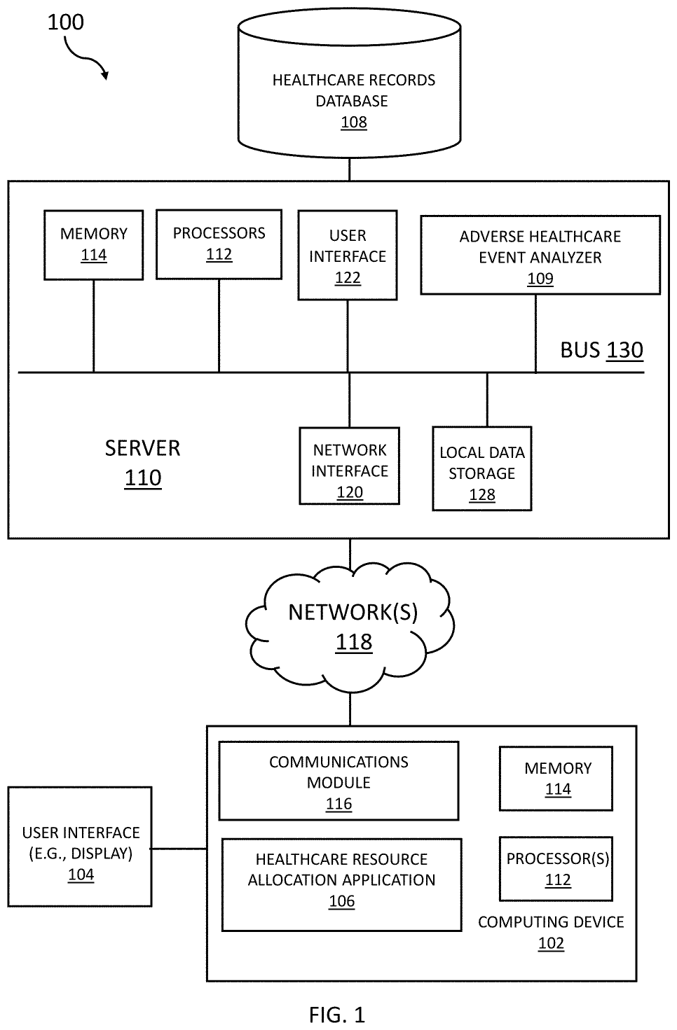

FIG. 1 illustrates a block diagram of a system 100 configured to determine and present risk indicators of patients to avoidable healthcare events in accordance with embodiments of the present disclosure. Referring to FIG. 1, the system 100 includes a computing device 102. The computing device 102 may be a desktop computer, laptop computer, smartphone, or the like. The computing device 102 includes a user interface 104 for presentation of indicators of risk of individuals to avoidable healthcare events in accordance with embodiments of the present disclosure. The user interface 104 in this example is a display that can be used to display a suitable representation (e.g., number or graphic) of an indicator of risk of one or more individuals to an avoidable healthcare event. Alternative to a display, the user interface 104 may be any other suitable electronic device integrated into the computing device 102 or operably connected to the computing device 102 for presenting a representation of an indicator of risk. For example, in the alternative the user interface 104 may be a speaker or printer. In addition, the computing device 102 may include other user interfaces for receipt of user input, such as a keyboard, a mouse, microphone, or the like.

The computing device 102 includes a healthcare resource allocation application 106 configured to correlate data associated with multiple individuals (e.g., patients) to an avoidable healthcare event for one or more of the individuals. The healthcare resource allocation application 106 is also configured to receive an indicator of risk and to present the indicator of risk to an operator. The healthcare resource allocation application 106 can use the user interface 104 to display the generated indicator of risk to the computing device's 102 operator. In accordance with embodiments, a server 110 can generate a model that relates the data to the avoidable healthcare event based on the correlation of the data to the avoidable healthcare event, and to apply the model to data of another individual to generate an indicator of risk of the other individual to the avoidable healthcare event. Further, the generated model may be a regression model or any other suitable model for use in analyzing the individuals' data by suitable statistical processes for estimating a relationship between the data and an identified avoidable healthcare event for one or more of the individuals. An example regression model is a linear regression model.

The server 110 can include a healthcare resource allocation analyzer 109 that can correlate data to an avoidable healthcare event for one or more of the individuals, generate a model that relates the data to the avoidable healthcare event based on the correlation of the data to the avoidable healthcare event, and apply the model to data of another individual to generate an indicator of risk of the other individual to the avoidable healthcare event. The analyzer 109 may be implemented by hardware, software, firmware or combinations thereof on the server 110. For example, the analyzer 109 may be implemented by one or more processors 112 and memory 114 of the server 110. The memory 114 may store instructions implemented by the processor(s) 112 for implementing functionality of the analyzer 109.

As an example, the application 106 may be configured to control a display to present to an operator an interface for interacting with the analyzer 106. The operator may use the application 106 for assessing one or more individual's risk of an avoidable event such as, but not limited to, hospitalization and emergency department visit. The operator may interact with the application 106 to suitably identify the one or more individuals. Subsequent to receiving input of the identifier(s) for the individual(s), the application 106 can generate indicator(s) of risk of the identified individual(s) to an avoidable healthcare event. The operator may also interact with the application 106 to specify the avoidable healthcare event.

In accordance with embodiments, the analyzer 109 at the server 110 may generate a regression model that relates data of multiple patients to an avoidable healthcare event (e.g., avoidable hospitalization) based on a correlation of that data to the event. In the example of FIG. 1, the data may be stored remotely in a healthcare records database 108. Example data stored in the database 108 includes, but is not limited to, diagnoses of the individuals, healthcare procedures of the individuals, medications of the individuals, utilization of healthcare services, demographic information, and geographic locations associated with the individual, and the like. The analyzer 109 at the server 110 may use some or all of the data to generate the regression model in accordance with embodiments of the present disclosure.

In an alternative example, the model may be generated at another computing device, such as another server. In this example, the other computing device has access to the database 108. The other computing device may also include an avoidable healthcare event analyzer configured to generate the model in accordance with embodiments of the present disclosure. Further, the analyzer at the other computing device may apply the model to data of another individual to generate an indicator of risk of the other individual to the avoidable healthcare event in accordance with embodiments of the present disclosure. In this case, the other computing device may communicate the indicator of risk to the computing device 102 for presentation to its operator.

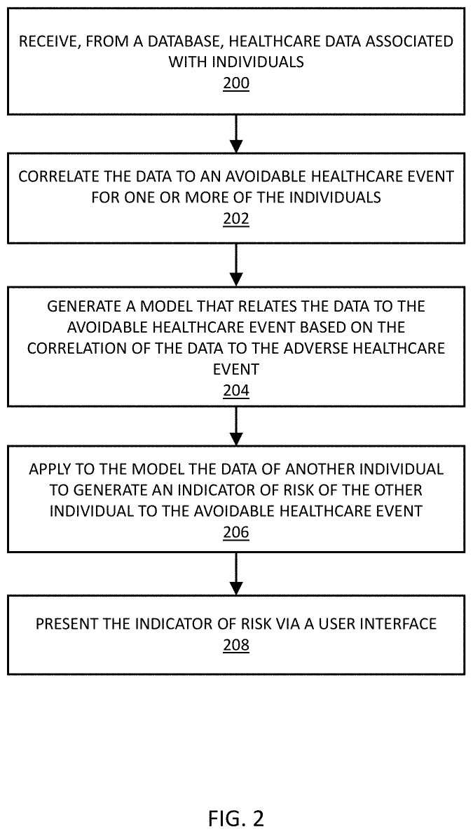

FIG. 2 illustrates a flow diagram of an example method for determining and presenting risk indicators of patients to avoidable healthcare events in accordance with embodiments of the present disclosure. The method may be implemented by any suitable computing device or computing devices working together. With regard to FIG. 2, the method is described as being implemented by the adverse healthcare event analyzer 109 of the server 110 and the application 106 of computing device 102.

Referring to FIG. 2, the method includes receiving 200, from a database, healthcare data associated with individuals. For example, the server 110 shown in FIG. 1 can identify healthcare records and request the identified healthcare records from the database 108. The server 110 may build a training dataset including demographic variables and clinical and functional indicators for a target population of individuals. For example, these variables and indicator may be collected from a combination of administrative claims data and in-person assessment tools. It is noted that the functionality of the analyzer 109 may be implemented on one or more computing devices (e.g., one or more servers).

It is noted that the server 102 can include a user interface 122, memory 124, one or more processors 126, and local data storage 128 for implementing its functionality. These components of the server 102 may suitably communicate with each other via a bus 130. The server 102 may check a requester's credential for requesting the data. In response to approval of the credentials, the server 102 may be able to access the requested data.

Now turning again to FIG. 2, the method includes correlating 202 the data to an avoidable healthcare event for one or more of the individuals. Continuing the aforementioned example, the analyzer 109 at the server 110 can correlate the received data to an avoidable event for an individual. For example, the analyzer 109 can match the data to records for the avoidable healthcare event (e.g., hospitalization and emergency facility visit, etc.) at a person level organized longitudinally.

The method of FIG. 2 includes generating 204 a model that relates the data to the avoidable healthcare event based on the correlation of the data to the avoidable healthcare event. Continuing the aforementioned example, the analyzer 109 can generate a discrete time survival model or other suitable model that relates the data to the avoidable healthcare event based on the correlation of the data to the avoidable healthcare event. The model can assign coefficient weights and significance scoring to each covariate.

The method of FIG. 2 includes applying 206 to the model the data of another individual to generate an indicator of risk of the other individual to the avoidable healthcare event. Continuing the aforementioned example, the analyzer 109 can apply the generated model to another individual or several other individuals to generate an indicator of risk of the individual(s) to the avoidable healthcare event. In an example, the analyzer 109 can use a discrete time survival model.

The method of FIG. 2 includes presenting 208 the indicator of risk via a user interface. Continuing the aforementioned example, the server 110 may communicate to the computing device 102 the indicator of risk. For example, the computing device 102 can include a communications module 116 configured to send the request to the server 110 via one or more communications networks 118 (e.g., internet and/or local area networks). The server 110 can receive the request via its network interface 120. In response to receipt of the request, an analyzer 106 of the server 102 may communicate the indicator of risk to the computing device 102 via the network(s) 118. Further, the application 106 can control the user interface 104 to present the indicator of risk. For example, the user interface 104 can display the indicator of risk. The indicator of risk can be used by a healthcare manager or other to assess risk and resources. Coefficients can be recalibrated with any significant policy, program, or population shift.

In accordance with embodiments, systems and methods disclosed herein can improve efficiency in the allocation of scarce healthcare coordination resources. If such resources are limited and the patients in a given healthcare practice panel differ in the benefit they would obtain through an intervention such as care coordination or the like, then patient outcomes can be optimized by focusing those care coordination resources on the patients for whom these resources can generate the most benefit. A model as disclosed herein can be used to rank attributed beneficiaries in each practice's panel based on their risk of experiencing an avoidable hospital event in order to assist in the identification and care coordination efforts for those high-risk individuals. Benefit can thereby be provided as the avoidance of a patient-specific adverse event. Many distinct adverse events are possible (ranging from disease onset to institutionalization to death), but given the emphasis on the reduction of unneeded utilization, the risk model can focus on potentially avoidable hospitalization or emergency department (ED) visits or similar adverse events. This can form the theoretical basis for the model presented herein, i.e. those individuals with the highest probability of incurring an adverse event are likely to benefit the most from an intervention with respect to that outcome. Through the dissemination of the model risk scores disclosed herein, an aim according to the present subject matter is to facilitate the identification of these individuals within each practice so that practices can allocate their interventions accordingly.

In accordance with embodiments, it can be important that the risk scores (i.e., indicators of risk) are as accurate as possible: ideally, the riskiest individuals as identified by the model have the highest actual likelihood of incurring an adverse event, and the individuals identified by the model as lowest risk have the lowest actual likelihood of incurring an adverse event. Due to the nature of the modeling problem—estimating the distribution of risk, rather than binary classification—it may not be appropriate to use the traditional Receiver Operator Characteristic curve as a measure of model fit. Instead, the utility of the model is assessed using concentration curves, which estimate the share of all avoidable hospital events occurring within the riskiest patients. Concentration curves can indicate, for example, that 50 percent of all patients who experience an avoidable hospital event are in the top 10 percent riskiest patients as estimated by the presently disclosed model. Concentration curves and month-by-month summary scores for the model are disclosed in examples provided herein.

It is important to note that the model's risk scores can use risk factors based on diagnoses, procedures, medications, utilization, demographics, and geographic factors in order to produce a practice-specific ranking of patient risk of an adverse event in the near future or a predetermined period of time. High medical expenditure can reflect a number of factors ranging from moderate utilization of high-cost procedures, high utilization of moderate-cost procedures, underlying morbidity, or geographic differences in treatment or referral practices. The risk score is designed to estimate, as closely as possible, event risk: that is, an individual's risk of an event in the following time period. The risk scores may be updated at set time periods and may use patient-level risk factor information current to the most recently available period of healthcare data in order to generate risk scores. Finally, by definition, adverse events are preventable through timely primary care and so, in principle, the identification and management of individuals at high risk of incurring an avoidable hospitalization may result in the avoidance of that particular hospitalization event. High medical expenditures, however, may reflect underlying morbidities that would necessitate utilization regardless of primary care intervention.

In order to illustrate the intended use of the risk scores, a clinical vignette has been created. For the sake of exposition, the patient panel consists of thirteen patients, each of which represents ten similar patients. Table 1 shows the patient panel, along with each patient's risk score and risk tier.

TABLE 1
Hypothetical Patient Panel

| Patient Name | Pre-AH Risk Score (%) | CMS Risk Tier |
|---|---|---|
| Patient 1 | 75% | Complex |
| Patient 2 | 15% | Complex |
| Patient 3 | 5% | Tier 4 |
| Patient 4 | 4% | Complex |
| Patient 5 | 2% | Tier 3 |
| Patient 6 | 1% | Tier 3 |
| Patient 7 | Less than 1% | Tier 2 |
| Patient 8 | Less than 1% | Tier 2 |
| Patient 9 | Less than 1% | Tier 1 |
| Patient 10 | Less than 1% | Tier 2 |
| Patient 11 | Less than 1% | Tier 1 |
| Patient 12 | Less than 1% | Tier 1 |
| Patient 13 | Less than 1% | Tier 1 |

Patients in this practice are listed in descending order of risk. Based on the most recently available period of risk factors spanning diagnoses, procedures, medications, utilization, demographics, and geographic information, in conjunction with risk coefficients derived from training data, Patient 1 (or, equivalently, the ten patients represented by Patient 1) has a 75 percent chance of incurring an adverse event in the near future. Patient 2 is the next riskiest, and has a 15 percent chance of incurring an adverse event. Patient 3 is the next riskiest, with a 5 percent chance. The distribution of risk is highly skewed: the majority of the practice's panel has less than 1 percent chance of incurring an avoidable hospital event, and all but two of the patients have under a 6 percent event risk.

An equal distribution of an intervention in this panel is unlikely to have a significant impact on patient outcomes: the low-risk individuals would be low-risk even without the intervention, and the high-risk individuals may require more resource-intensive interventions in order to experience improvement in outcomes. The risk scores, used in conjunction with provider clinical guidance, can assist practices and providers with a more efficient and impactful allocation of their care management efforts.

In accordance with embodiments, the model may be used to display to practices and providers the top actionable risk factors underlying each patient's risk of incurring a future adverse event. An intention is to augment the information provided to practices in order to further facilitate patient-specific interventions. For example, in addition to a risk score of 3.2 percent for a particular patient, care managers may also be able to see on the dashboard that the patient (for example) meets the clinical criteria for diabetes and heart failure and incurred a claim for insulin within the past year (in descending order of contribution to risk). While that patient may also have had other salient risk factors—for example, meeting the clinical criteria for depression—the system may only display the most predictive, intervene-able risk factors in order to allow care mangers to focus their attention on the most pressing patient needs.

Reasons for risk can be based on the underlying risk factor coefficients, which are derived from a training phase of the model. It is important to note that these coefficients may not necessarily have a causal interpretation: they only capture the strength of association between a given risk factor and the risk of incurring a future avoidable hospital event. For example, if the risk factor coefficient for diabetes is positive, then that may mean that having diabetes causes an increased risk of adverse events; however, it may also mean that having diabetes is only correlated with some unobserved factor that causes an increased risk of adverse events. While these risk factors do not have a strictly causal interpretation, they are intended to provide care managers with a useful starting point from which to address specific patient needs.

In order to operationalize the identification of reasons for risk, the model may be configured such that a higher level of a given risk factor is associated with greater risk of incurring an adverse event. Consider the example of flu vaccinations: there is evidence that influenza and/or pneumococcal vaccinations reduce the risk of hospitalization for various events in various populations. This implies that receipt of a flu vaccination may be negatively associated with the risk of incurring an adverse event. This risk factor, then, can be configured to be 1 if the individual has not received a flu vaccination, and 0 if the individual has received a flu vaccination.

In accordance with embodiment, there can be two criteria for determining which risk factors to include. For example, first if there is strong a priori empirical evidence that certain risk factors—again, like having had a flu vaccination—have a negative association with the risk of incurring an avoidable hospitalization, then the variable can be configured accordingly. Second, for example, if the impact of a given risk factor on the risk of incurring an avoidable hospital event is ambiguous, then the Andersen Behavioral Model of health services utilization can be applied to guide configuration of the logic. The Andersen model posits that health services utilization is a function of predisposing, enabling, and need factors.

While the baseline model contains approximately 200 risk factors, only a subset of these are included in the pool of potential reasons for risk for reasons of statistical interpretation and clinical utility. Most non-binary and non-count risk factors are excluded because these cannot easily be translated into reason for risk contributions for lack of a meaningful reference group. Additionally, based on the feedback from stakeholders, risk factors are excluded that are not potentially modifiable; that is, for which the effects cannot be meaningfully modified by clinical intervention (like, for example, area income). Finally, risk factors that are not positive and statistically significant are also excluded.

Consider the following illustrative example. Suppose that the model contains only three risk factors: a flag for diabetes, the number of recent avoidable hospitalizations, and a flag for heart failure. In this example, the coefficients for these three risk factors are 0.1, 0.08, and 0.07, respectively. The coefficient for diabetes represents the increase in risk of avoidable hospitalizations associated with having diabetes (relative to not having diabetes), holding all other factors constant. The coefficient for the number of avoidable hospitalizations reflects the added risk associated with one additional previous avoidable hospitalization, and the coefficient for heart failure reflects the added risk associated with having heart failure (relative to not having heart failure), again holding all other factors constant.

It is important to note that these risk coefficients may be marginal effects; that is, the additional risk due to, for example, a patient having one additional previous avoidable hospitalization. In order to translate these marginal effects to reason for risk contributions, the system can multiply each marginal estimate by the level of that risk factor for each individual. Thus, if an individual has four previous avoidable hospitalizations, then the risk contribution of avoidable hospitalizations is 4*0.08=0.32. This risk contribution may still be interpreted relative to a reference category: in this case, that of individuals with no history of avoidable hospitalizations. More broadly, the risk contribution may be interpreted relative to individuals without that particular risk factor.

Suppose that, in this example, there are four patients in a program. Patient 1 has diabetes, no history of avoidable hospitalization, and heart failure. Patient 2 does not have diabetes, has no history of avoidable hospitalization, and has heart failure. Patient 3 has diabetes, four prior avoidable hospitalizations, and does not have heart failure. Finally, patient 4 does not have diabetes, has one previous avoidable hospitalization, and has heart failure. This information is presented in Table 2, below.

TABLE 2
Hypothetical Reason for Risk Example

| Patient | | Diabetes * | # | # AH * | Heart | Heart Failure * |
|---|---|---|---|---|---|---|
| ID | Diabetes | Coefficient | AH | Coefficient | Failure | Coefficient |
| 1 | 1 | 0.1 | 0 | 0.0 | 1 | 0.07 |
| 2 | 0 | 0.0 | 0 | 0.0 | 1 | 0.07 |
| 3 | 1 | 0.1 | 4 | 0.32 | 0 | 0.0 |
| 4 | 0 | 0.0 | 1 | 0.08 | 1 | 0.07 |

In this example, the top reason for risk for Patient 1 is diabetes: this risk factor yields the largest positive contribution (risk factor level * coefficient) among all the risk factors for that individual. For Patient 2, the top reason for risk is heart failure; for patient 3, the top reason for risk is the history of avoidable hospitalizations; and for patient 4, the top reason for risk is the history of avoidable hospitalizations. The second reason for risk is calculated analogously: it is the second highest contribution of (risk factor level * coefficient) for each individual. All other reasons for risk are estimated in a similar fashion.

In a dashboard in accordance with embodiments, users can also see the contribution of each risk factor category (Condition, Demographic, Pharmacy, Utilization, and Environmental) in percentage terms. These are intended to provide a high-level description of the contribution of various types of risk factors that are positive and significant for an individual. The contribution for a given category is calculated as the sum of (risk factor level * coefficient) for all reasons for risk in that category, divided by the sum of (risk factor level * coefficient) for all positive, statistically significant reasons for risk. This is an important point: an individual's overall risk is a function of all risk factors, including those that are not included as potential reasons for risk. The category contributions, however, are only interpretable relative to the reason for risk factor pool, which is restricted to the operationalizable, modifiable risk factors.

In accordance with embodiments, systems and methods disclosed herein may utilize a database having healthcare claims, supplemented with various publicly available environmental data sets used to generate the environmental risk factors. Example data sources are disclosed herein.

The majority of the risk factors in the systems disclosed herein may be derived from various government healthcare claims files. Each period, the system may receive this data. Additionally, the system may receive beneficiary attribution files and practice rosters each quarter.

Upon receipt of the claims files, the system can initially perform automated data validity checks in order to assess the integrity of the data files, followed by a data reduction step that subsets the claims files against the beneficiary attribution file. The resulting files retain the raw claims data that are inputs to the risk factor feature engineering process, but discard the claims for individuals that are not in the population.

In accordance with embodiments, the model may be created using suitable risk factors. Example risk factors are disclosed herein.

In order to control for environmental factors that may affect patients' probabilities of incurring adverse events, the risk model includes a rich set of area-level covariates derived from publicly available sources. Based on the geographic location, each attributed beneficiary can be linked to environmental characteristics in his or her residential area.

In accordance with embodiments, various risk factors have been identified and operationalized to be included in the risk model. While some of these risk factors are eliminated in the variable selection step, this process is data-driven, and all risk factors are included in the pool of potential risk factors to be used in the model. A high-level description of risk factors is provided herein.

Various risk factors have been identified for use with systems and methods disclosed herein. For example, risk factors based on institutional claims cover information on admissions over the past 12 months; nursing home stays over the past 12 months; and certain procedures. Additionally, the institutional claims can be used in order to construct an adverse event outcome, as well as the diagnostic condition flags. These condition flags rely on diagnostic information from institutional and physician claims in order to generate individual-level risk factors that represent underlying disease states.

In another example, risk factors based on physician claims cover utilization of certain services (such as vaccinations, lab tests, or J-code procedures), place of service (for example, urgent care or rural health clinic), and provider specialty (for example, endocrinology or oncology). Also, risk factors were created to capture an individual's primary care utilization and continuity of care. Also, the physician claims are used in order to construct the adverse event outcome, as well as the diagnostic condition flags.

In another example, using pharmacy claims, the system can flag utilization of drugs identified as potential risk factors for adverse events. In order to capture compound drugs, which are drugs that contain multiple active ingredients, the system can rely largely on text-based, “contains”-type searches of the FDA's “National Drug Code Directory.” See Table 3 below for the primary search strategy, as well as for a list of the substances flagged.

TABLE 3
Primary Search Strategy for MDPCP Pharmacy Risk Factors

| Risk Factor | Primary Search Method in NDC | Substances Flagged |
|---|---|---|
| Rivaroxaban | Nonproprietary name contains | RIVAROXABAN |
| use | “RIVAROXABAN” | |
| Losartan use | Substance name contains | LOSARTAN POTASSIUM; LOSARTAN POTASSIUM and |
| | “LOSARTAN” | HYDROCHLOROTHIAZIDE |
| Warfarin use | Substance name contains | WARFARIN SODIUM |
| | “WARFARIN” | |
| Cilostazol use | Substance name contains | CILOSTAZOL |
| | “CILOSTAZOL” | |
| Insulin use | Substance name or nonproprietary | INSULIN ASPART; INSULIN DEGLUDEC; INSULIN |
| | name contains “INSULIN” and | DEGLUDEC and LIRAGLUTIDE; INSULIN DETEMIR; |
| | marketing category name does not | INSULIN GLARGINE; INSULIN GLARGINE and |
| | contain “UNAPPROVED” | LIXISENATIDE; INSULIN GLULISINE; INSULIN HUMAN; |
| | | INSULIN LISPRO |
| Statin use | Drug Class contains “HMG-CoA | SIMVASTATIN; LOVASTATIN; PITAVASTATIN; |
| | Reductase Inhibitor” or substance | ROSUVASTATIN CALCIUM; PRAVASTATIN SODIUM; |
| | name contains “ROSUVASTATIN | FLUVASTATIN SODIUM; PITAVASTATIN CALCIUM; |
| | CALCIUM” | ATORVASTATIN CALCIUM; ATORVASTATIN CALCIUM |
| | | TRIHYDRATE; ATORVASTATIN CALCIUM PROPYLENE |
| | | GLYCOL SOLVATE; EZETIMIBE and SIMVASTATIN; |
| | | NIACIN and LOVASTATIN; SIMVASTATIN and NIACIN; |
| | | AMLODIPINE BESYLATE and ATORVASTATIN |
| | | CALCIUM; AMLODIPINE BESYLATE and |
| | | ATORVASTATIN CALCIUM TRIHYDRATE |
| Leukotriene | Drug class contains “Leukotriene | MONTELUKAST; MONTELUKAST SODIUM; |
| Receptor | Receptor Antagonist” | ZAFIRLUKAST |
| Modifier use | | |
| Beta Blocker | Substance Name contains | CARVEDILOL; CARVEDILOL PHOSPHATE; |
| use | “METOPROLOL” or “CARVEDILOL” | METOPROLOL SUCCINATE; METOPROLOL TARTRATE; |
| | | METOPROLOL TARTRATE and |
| | | HYDROCHLOROTHIAZIDE; METOPROLOL SUCCINATE |
| | | and HYDROCHLOROTHIAZIDE |
| Oral | Drug class contains “Corticosteroid” | BUDESONIDE; CORTISONE ACETATE; DEFLAZACORT; |
| Corticosteroid | and route is “ORAL” and dosage | DEXAMETHASONE; HYDROCORTISONE; |
| use | form contains either “CAPSULE” or | METHYLPREDNISOLONE; PREDNISOLONE; |
| | “TABLET” and marketing category | PREDNISOLONE SODIUM PHOSPHATE |
| | does not contain “UNAPPROVED” | |
| Antidiabetes | Substance name contains “FLOZIN”, | ALOGLIPTIN BENZOATE; ALOGLIPTIN BENZOATE and |
| Medication | “GLIPTIN”, “THIAZOLIDINEDIONE”, | METFORMIN HYDROCHLORIDE; ALOGLIPTIN |
| | “ROSIGLITAZONE”, or | BENZOATE and PIOGLITAZONE HYDROCHLORIDE; |
| | “PIOGLITAZONE” | CANAGLIFLOZIN; CANAGLIFLOZIN and METFORMIN |
| | | HYDROCHLORIDE; DAPAGLIFLOZIN; DAPAGLIFLOZIN |
| | | PROPANEDIOL; DAPAGLIFLOZIN PROPANEDIOL and |
| | | METFORMIN HYDROCHLORIDE; DAPAGLIFLOZIN and |
| | | SAXAGLIPTIN HYDROCHLORIDE; EMPAGLIFLOZIN; |
| | | EMPAGLIFLOZIN and LINAGLIPTIN; EMPAGLIFLOZIN |
| | | and METFORMIN HYDROCHLORIDE; ERTUGLIFLOZIN |
| | | PIDOLATE; ERTUGLIFLOZIN PIDOLATE and |
| | | METFORMIN HYDROCHLORIDE; ERTUGLIFLOZIN |
| | | PIDOLATE and SITAGLIPTIN PHOSPHATE; |
| | | LINAGLIPTIN; LINAGLIPTIN and METFORMIN |
| | | HYDROCHLORIDE; METFORMIN HYDROCHLORIDE |
| | | and PIOGLITAZONE HYDROCHLORIDE; |
| | | PIOGLITAZONE; PIOGLITAZONE HYDROCHLORIDE; |
| | | PIOGLITAZONE HYDROCHLORIDE and GLIMEPIRIDE; |
| | | PIOGLITAZONE HYDROCHLORIDE and METFORMIN |
| | | HYDROCHLORIDE; ROSIGLITAZONE MALEATE; |
| | | SAXAGLIPTIN HYDROCHLORIDE; SAXAGLIPTIN |
| | | HYDROCHLORIDE and METFORMIN |
| | | HYDROCHLORIDE; SITAGLIPTIN PHOSPHATE; |
| | | SITAGLIPTIN PHOSPHATE and METFORMIN |
| | | HYDROCHLORIDE |
| Oral Antibiotics | Substance name contains | See Appendix 2. |
| | prescription names in NCQA | |
| | “Antibiotics of Concern” and “All | |
| | other Antibiotics” and route is | |
| | “ORAL”- See Appendix 2. | |

Several example risk factors include individual-level demographic and socioeconomic factors that are unavailable in healthcare claims data (for example, marital status). Consequently, corresponding area-level risk factors (for example, the percentage of the population aged 15+ that is currently married) can be included in the risk model in order to proxy for the unobserved individual-level variables. Other environmental risk factors (for example, the area poverty rate) can capture the social determinants of health: the neighborhood conditions in which people live and age that may affect health outcomes.

Other example risk factors include: an indicator for frailty, an indicator for original Medicare eligibility due to a non-age related reason, an indicator for durable medical equipment use within the past year, the number of ED visits in the past 6 months, an indicator for sickle cell anemia, ZIP code pollution level, ZIP code walkability, and ZIP code pharmacy density.

Methodologically, the system may rely on a discrete time survival model that uses current values of procedural, diagnostic, utilization-based, pharmacy, demographic, and environmental risk factors to predict the likelihood that an individual incurs an avoidable hospitalization or ED visit in the following month. The parameter estimates generated in the model training can be subsequently used to generate individual risk predictions in the scoring step.

An outcome measure can be a 0/1 indicator variable denoting whether an individual incurred an avoidable hospitalization or ED visit in a given month. In order to construct this measure, the system can rely on various technical definitions provided by the Agency for Healthcare Research and Quality (AHRQ) as part of its prevention quality indicator (PQI) measures. Diagnosis codes from inpatient and ED claims can be used to flag the following conditions, which are the basis for the composite PAH flag:
PQI #1: Diabetes Short-Term Complications
PQI #3: Diabetes Long-Term Complications
PQI #5: COPD or Asthma in Older Adults
PQI #7: Hypertension
PQI #8: Heart Failure
PQI #10: Dehydration
PQI #11: Bacterial Pneumonia
PQI#12: Urinary Tract Infection
PQI #14: Uncontrolled diabetes
PQI #15: Asthma in Younger Adults
PQI #16: Lower-Extremity Amputation among Patients with Diabetes

This can be implemented in the model as an indicator variable at the person-month level. If an individual incurs at least one avoidable hospitalization or ED visit in a given month, then that person receives a value of 1 for this variable—and 0 otherwise.

Avoidable hospitalization/ED visits are recurrent events with time-dependent covariates. Accordingly, the system may be operationalized as a discrete-time survival model that uses the current month of risk factors in order to predict avoidable hospitalization/ED visits in the following month. The model can use month as a time unit—instead of, for example, weeks or years—in order to balance the increased model accuracy obtained by a more granular time unit with the increased prediction error due to rare events.

The raw healthcare claims data span three years, or 36 person-months for individuals with full coverage. Since the model estimates the risk of incurring an avoidable hospitalization in the next month, however, it may not be possible to use the most recently available month of risk data in the training model (since the next month's avoidable hospitalizations have not been realized at this point). Therefore, the training data are based on underlying data covering 35 person-months per attributed patient with full coverage. While, in general, a reduction in sample size can adversely impact the statistical precision of the risk factor estimates, lagged predictors are used for three reasons.

In accordance with embodiments, the statistical model can be trained on an 80 percent sample of an analytical person-month data set. The functional form of the statistical model is:

$$log ( \frac{{p}_{i} ( t )}{1 - {p}_{i} ( t )} ) = φ ( t ) + β {X}_{i} ( t - 1 ) + {Ω V}_{i}$$

φ(t) is a cubic function of time, accounting for the time effect
β and Ω are the vectors of model parameters to be determined by training data
Xᵢ(t−1) is a vector of patient i's time-dependent features in the previous month
Vᵢ is a vector of patient i's time-independent features
pᵢ(t) is the probability of avoidable hospitalization or ED visit of patient i at time t (i.e., the month following the realization of the risk factors)
t is duration of time in months
   counting start from the first month of available data if the patient is in coverage longer than three years, or
   counting from the coverage start month if the patient's coverage start is within three years
      The statistical model can use six types of risk factors: diagnostic, pharmacy, procedural, utilization-based, geographic, and demographic. It is important to note that not all risk factors are available for every person-month. The system can use a twelve-month lookback period for most of the time-varying risk factors, implying that an individual with, for example, five months of claims history will have incomplete information in her risk factors: if this individual truly has glaucoma, then it is possible that she will not amass the claims history by month five that meets the qualifications required for a glaucoma flag in our model. Furthermore, while most individuals in the data have valid geographic indicators that link to the environmental risk factor data set, several hundred beneficiaries do not, and therefore receive no environmental risk factors. Table 4 presents the risk factor availability, depending on claims history and the availability of geographic data.

TABLE 4
Availability of Risk Factors for Scoring

| | | At Least 12 Months of Claims History | |
|---|---|---|---|
| | | Yes | No |
| Availability of | Yes | Dx/Rx/Proc/Util/Geo/Demo | Geo/Demo |
| Geographic Risk | No | Dx/Rx/Proc/Util/Demo | Demo |
| Factors | | | |

Risk factor availability is an issue for the “scoring” step, in which risk scores are assigned to every individual based on the parameter estimates derived in the training step. For example, suppose that the vector of estimated coefficients from the logistic regression is as follows in Table 5.

TABLE 5
Example Risk Model Coefficients

| Risk Factor | Value for individual i |
|---|---|
| Asthma Flag | .1 |
| . . . | |
| ZIP Code Income | −.00001 |
| . . . | |
| Age | .02 |

These hypothetical risk factor coefficients indicate that, as expected, if an individual meets the clinical criteria for asthma, the risk of avoidable hospitalization is higher; if he or she lives in a ZIP code with higher income, the risk is lower; and if he or she is older, the risk is higher. The scoring step can apply this vector of coefficients to the “person-now;” that is, the current month for each individual. Individual i's predicted probability of incurring an avoidable hospitalization in the next month, then, will be scored as follows:

$${Risk}_{i} = \frac{{e}^{.1 * {Asthma}_{i} + . . . - .00001 * ZIP Code {Income}_{i} + . . . + .02 * {Age}_{i}}}{1 + {e}^{.1 * {Asthma}_{i} + . . . - .00001 * ZIP Code {Income}_{i} + . . . + .02 * {Age}_{i}}}$$

Suppose that these variables (Asthma Flag, ZIP Code Income, and Age) are the only three risk factors in the model. Furthermore, suppose that individual i has the following characteristics:

TABLE 6
Example Risk Factors

| Risk Factor | Value for individual i |
|---|---|
| Asthma Flag | 1 |
| ZIP Code Income | $55,000 |
| Age | 66 |

This hypothetical individual has asthma, lives in a ZIP Code in which the median income is $55,000, and is 72 years old. Then, that individual's risk of an avoidable hospital event in the following month is

$$\frac{{e}^{( . 1 * 1 - .00001 * 55 , 0 \; 00 + .02 * 66 )}}{1 + {e}^{( . 1 * 1 - .000 \; 01 * 55 , 0 \; 00 + .02 * 66 )}} = 70.47 percent .$$

Suppose, however, that this individual is newly eligible for Medicare and does not have sufficient claims history to meet the criteria for an asthma flag (anything under 12 months). In this instance, the individual might truly have asthma as an underlying disease state, but this is not observable. The individual's risk factors, then, are:

TABLE 7
Example Risk Factors with Missing Information

| Risk Factor | Value for individual i |
|---|---|
| Asthma Flag | NOT OBSERVED |
| ZIP Code Income | $55,000 |
| Age | 66 |

If the model's coefficients are applied only to the risk factors that are observed, then this individual's estimated risk is 68.35 percent. By failing to account for the risk factors that are not present in the model, the risk of incurring an avoidable hospital event is underestimated for individual i.

In one example, this issue may be solved by estimating four different models based on the risk factors that are available for each group. This allows the risk factors that are present to “compensate,” to a certain extent, for the risk factors that are missing due to data availability. For example, suppose that an individual lacks sufficient claims history to generate diagnostic risk factors but does have the following demographic risk factors: age, gender, and race. If gender is correlated with the unobserved diagnostic risk factors (if, for example, female beneficiaries are more likely to experience chronic conditions than male beneficiaries), then the coefficient for the “gender” risk factor will capture this correlation, and thus represent the marginal impact of being female and the portion of unobserved diagnostic risk factors that is correlated with gender. Consequently, if female beneficiaries are more likely to experience chronic conditions than male beneficiaries, then the risk factor coefficient for “gender” will be larger in the models without diagnostic risk factors than in the models with diagnostic risk factors. By allowing observed risk factors to capture some of the predictive power of unobserved risk factors, the loss in predictive power due to missing data is minimized.
Model 1: use Rx/Dx/Util/Proc/Geo/Dem risk factors
Model 2: use Geo/Dem risk factors
Model 3: use Rx/Dx/Util/Proc/Dem risk factors
Model 4: use Dem risk factors

The four models can be trained on the subset of person-months for which all risk factors are complete (that is, person-months with at least 12 months of claims history and a valid ZIP code), and include the following sets of risk factors (analogous to the four partitions of the person-month sample):
Model 1: use Rx/Dx/Util/Proc/Geo/Dem risk factors
Model 2: use Geo/Dem risk factors
Model 3: use Rx/Dx/Util/Proc/Dem risk factors
Model 4: use Dem risk factors
   Variable selection can improve the performance of predictive models by reducing prediction variance and increasing generalizability. The system can perform this in two steps: first, the team selected initial risk factors based on an extensive literature review, which screened over 3,300 articles and ultimately selected 211 published, peer-reviewed papers from which to extract risk factors. This generated a pool of roughly 190 risk factors. Additionally, the system can use stepwise selection in the multivariable logistic model in order to remove insignificant predictors from the model before adding significant predictors.

In accordance with embodiments, the risk factors enter the model additively: that is, if an individual has both diabetes and heart failure diagnostic flags, then his or her risk score will reflect the risk coefficient for the diabetes flag, plus that of the heart failure flag. It is possible, however, that there is additional risk due to the fact of the beneficiary having both conditions, over and above the sum of the risks of having each condition. In embodiments, the model can identify and include these interaction variables.

The four risk models above can be trained on the subset of data with at least twelve months of claims history and full geographic data in order to estimate the vectors of coefficients for the risk factors in each model. These coefficients are described hereinbelow for Model 1 as odds ratios. Then, using the most recently available month of risk factors (that is, the “person-now” data set), individuals are scored using the model coefficients that correspond to the risk factors available in the person-now data set.

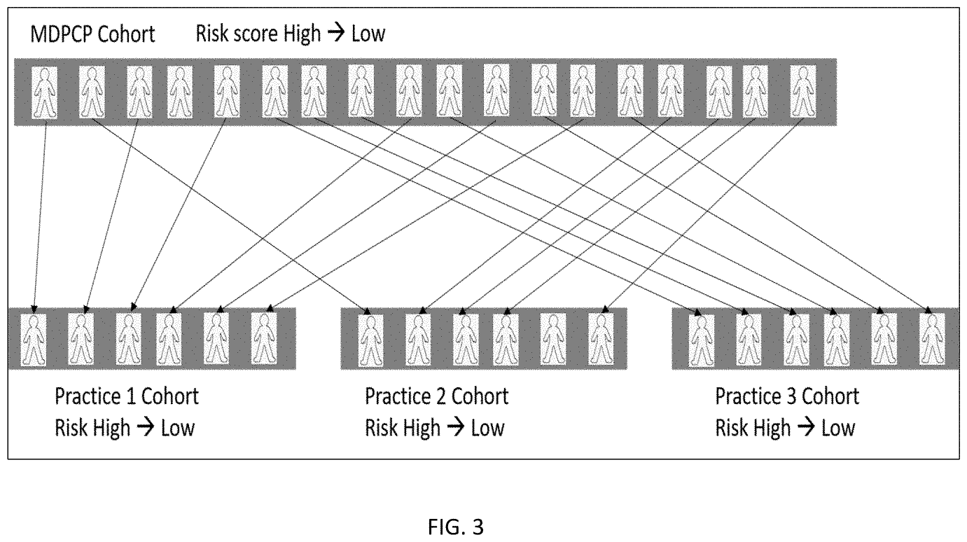

The system's model can generate risk scores for the entire population, but individual practices may receive only practice-specific risk scores. This has the consequence that, if a practice contains disproportionately high-risk patients, and another practice contains disproportionately low-risk patients, then the riskiest patients within each practice may differ in their absolute risk. FIG. 3 presents a diagram of this point. Particularly, FIG. 3 depicts a diagram showing stratification of a population of individuals by practices/physicians.

In accordance with embodiments, the system can train the model once per quarter. This entails creating the risk factors from the raw claims data and estimating the four models. The resulting risk factor coefficients can then be used to score the cohort once per month. This includes creating the risk factors from the raw claims data for the most recent month of claims history and then applying the most recent set of model coefficients.

For example, suppose that the four models are trained on Jan. 1, 2019, Apr. 1, 2019, Jul. 1, 2019, and Oct. 1, 2019, and that the previous month of claims data are available on the first day of the following month (so, in this example, claims data for June 2019 are available on Jul. 1, 2019). The model training generates risk factor coefficient estimates—one estimate for each risk factor—and these coefficient estimates are applied to the most recent set of risk factors in order to generate risk scores. For example, the coefficients estimated in the Jul. 1, 2019 training can be used with the June 2019 risk factors in order to predict risk of avoidable hospitalization in July 2019 (which, remember, has not yet been observed as of the Jul. 1, 2019 training date). These same risk factor coefficient estimates will be used with the July 2019 risk factors to predict August 2019 avoidable hospitalization and with August 2019 risk factors to predict September 2019 avoidable hospitalizations. Then, since the model is re-trained on Oct. 1, 2019, September 2019 risk factors can be used with the new training model coefficients to predict avoidable hospitalizations in October 2019, and so on.

It is possible that the risk predictions may fall in accuracy as the training data model coefficients “fall behind” the person-now scoring data in time. For example, it is possible that, for the Jul. 1, 2019 model training, the predictions are most accurate for July 2019 avoidable hospital events and then become less accurate for August 2019, and even less accurate for September 2019, since the training model coefficients become more removed from the current underlying data generating process. It is not believed that this represents a significant threat to the model's predictive accuracy. Any systematic bias may be the result of underlying structural changes in the relationship of certain risk factors to the risk of incurring an avoidable hospital event, which seems unlikely to occur in a three-month window.

It is also possible that the latest month of claims for a given training data set may not be complete: for example, suppose that data received in July 2019 contains claims only through mid-June 2019. In this instance, the system can still use these June 2019 risk factors in order to score the cohort in July 2019, for two reasons. First, since all of the time-variant risk factors include at least 12 months of lookback, there is relatively low month-to-month variation; consequently, there is a relatively low chance of failing to include salient risk factors as a result of the missing data from the second half of June 2019. Second, it is imperative for the utility of the risk scores that the system model uses the most recently available risk factors.

However, incomplete data for the final month of claims may introduce bias into the training model risk coefficients. To continue the example above, the training data can include beneficiaries' risk factors from May 2019, which are used to predict avoidable hospital events in June 2019. Since, in this example, claims information from late June 2019 will be missing, it will appear in the analytic data set as if most individuals did not incur an avoidable hospitalization in this month. Thus, in the extreme, May 2019 risk factors can be used to model all zeros for June 2019 outcomes, which would bias the risk factor coefficients toward zero. Given that 24 months of data are used in order to train the model, this is unlikely to meaningfully affect the training coefficients. The system can monitor this and, if needed, can train the model only on the months of data for which we have full information for the outcome variable. To finish this example, May 2019 risk factors (and June 2019 outcomes) may not be included in the training model.

As part of the ongoing development process for the model, internal testing was conducted on the utility of nonlinear modeling to predict avoidable hospital events in the cohort. The goal of the testing was to assess whether nonlinear modeling would improve model performance sufficiently to justify the development costs and reduced interpretive intuition that would result from deploying the nonlinear model into production.

The question of how much improvement in model fit is “enough” is inherently subjective. A decision-theoretic framework was used to guide this decision: given that development time is costly and the coefficients of non-linear models can be difficult to interpret, how best to allocate development effort in order to improve the model performance? In general, a model's predictive performance may be improved in two ways: either by adding new information to the model in the form of new risk factors, or by using existing information in different ways (for example, by adopting a different modeling methodology). In the June 2020 re-training, eight new risk factors were added to the model: an indicator for frailty, an indicator for original Medicare eligibility due to a non-age related reason, an indicator for DME use within the past year, the number of ED visits in the past 6 months, an indicator for sickle cell anemia, ZIP code pollution, walkability, and pharmacy density. This led to an increase in the Model 1 holdout sample C-statistic of 0.0051, and an increase in the holdout sample Gini score of 0.0107. These improvements in model performance were used as a baseline for gauging the added value of developing a nonlinear prediction model: in order to further pursue nonlinear modeling, the internal testing would need to demonstrate an increase in either the C-statistic or Gini score by at least 0.0051 or 0.0107, respectively.

Two separate nonlinear modeling methods were evaluated: neural network modeling and the inclusion of interaction terms into the baseline model. Both strategies are described hereinbelow.

Regarding neural network modeling, it is noted that neural networks are machine learning models that can, in principle, approximate any underlying function. These models estimate highly nonlinear and complex relationships between risk factors and the target variable to be predicted (in our case, avoidable hospital events), and then use these relationships in order to generate predictions. Researchers have documented that neural networks improve predictive models relative to logistic regression in certain contexts, but at the cost of considerable computational time and a loss of modeling transparency.

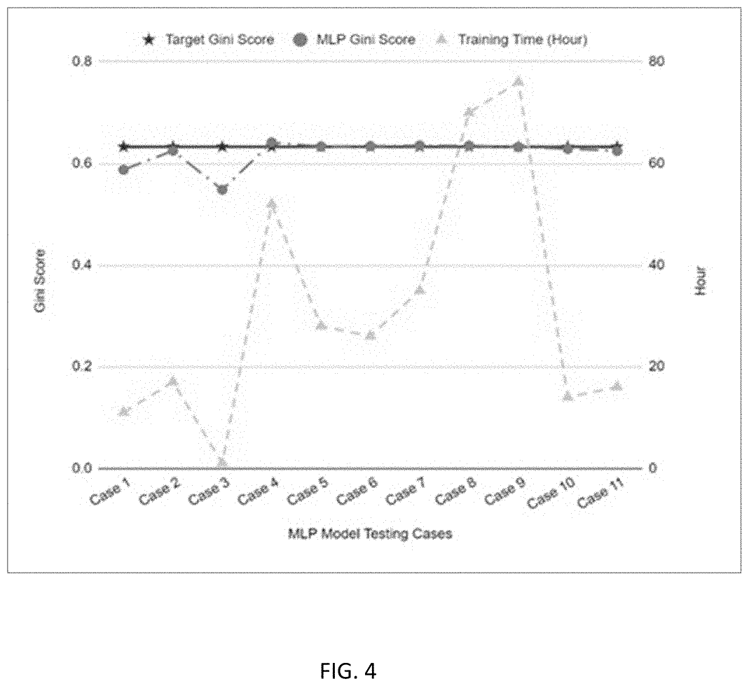

Eleven different neural networks were tested with varying combinations of hyperparameters (that is, technical aspects of neural network models). The Multi-Layer Perceptron (MLP) model was used. This model is a standard neural network model that is available in SAS. Results of this testing is summarized in FIG. 4, which is a graph. It was found that none of these eleven tests resulted in a substantially improved model performance, and most entailed significant computational costs. The best model performance was obtained in Case 4, which resulted in a 0.0085 increase in Gini score relative to baseline. However, this did not meet our internal standard for further development.

Regarding interaction terms, a model fit was assessed that would result from including interaction terms in the model. Interaction terms capture accumulative effects: for example, in addition to avoidable hospital event risk due to Heart Failure and risk due to COPD, it is possible that there is additional risk that accrues to individuals with both Heart Failure and COPD. For this test, we included various sets of interaction terms into the baseline model risk factors and assessed the resulting increase in C-statistic.

Given that the baseline model contains approximately 200 risk factors, it was computationally prohibitive to include all possible combinations of interaction terms; therefore, a machine learning decision tree model was estimated in order to determine the most “important” variables in our baseline model and then used these variables to create interaction terms. It was determined these variables to be the number of avoidable hospital events in the previous year, the number of ED visits in the previous six months, the total number of medications, an indicator for having heart failure, an indicator for having COPD, and an indicator for having diabetes with complications.

All fifteen possible two-way interactions were created from these six covariates, and including various combinations of these two-way interactions in our baseline model. Approximately 275 regressions were run and recorded the resulting C-statistic from each regression model. It was found that, at most, the C-statistic rose by 0.0017.

In experiments, a model according to embodiments of the present disclosure was applied to data from a period of May 2017 to April 2020. The results are presented herein. For example, Table 8 presents risk factor coefficient estimates for Model 1 for the training performed in June 2020. This model includes all six types of risk factors: diagnostic, pharmacy, procedural, utilization-based, geographic, and demographic. The risk factors in Table 8 are those that were included in the final model. All other risk factors were eliminated in the variable selection step due to insufficient predictive power. Note that the risk factor coefficients are presented as odds ratios, which can be interpreted in terms of a multiplicative impact: for example, an odds ratio of 1.05 indicates that if that risk factor were to increase by one unit, then the risk of incurring an avoidable hospitalization would increase by 5 percent.

TABLE 8
Risk Model Odds Ratios for Model 1

| Risk Factor | Odds Ratio |
|---|---|
| Prior hospitalization discharge status-other (relative to no | 2.246 |
| prior admission) | |
| CCW indicator for chronic obstructive pulmonary disease | 1.618 |
| (COPD) and bronchiectasis | |
| Indicator for retinopathy | 1.540 |
| Indicator for hospice enrollment | 1.448 |
| Prior hospitalization admission type-emergency (relative | 1.422 |
| to no prior admission) | |
| Indicator for original Medicare eligibility for a non-age | 1.415 |
| related cause | |
| CCW indicator for heart failure | 1.409 |
| Number of avoidable hospitalizations | 1.382 |
| Beneficiary race-Black | 1.357 |
| Indicator for urinary tract infection | 1.338 |
| Prior hospitalization admission type-urgent (relative to no | 1.296 |
| prior admission) | |
| Indicator for dual eligibility with Medicaid | 1.268 |
| Indicator for insulin use | 1.267 |
| CCW indicator for hypertension | 1.247 |
| CCW indicator for tobacco use | 1.244 |
| Indicator for durable medical equipment (DME) use | 1.220 |
| Indicator for arrhythmia | 1.210 |
| CCW indicator for chronic kidney disease | 1.208 |
| Indicator for fluid and electrolyte imbalance | 1.205 |
| CCW indicator for intellectual disabilities and related | 1.199 |
| conditions | |
| Indicator for problems with care provider dependency | 1.193 |
| Discontinuity of primary care-Proportion | 1.191 |
| CCW indicator for asthma | 1.180 |
| Indicator for metastatic cancer | 1.167 |
| Indicator for no statin use | 1.154 |
| CCW indicator for lung cancer | 1.153 |
| CCW indicator for ischemic heart disease | 1.152 |
| CCW indicator for pressure and chronic ulcers | 1.144 |
| CCW indicator for diabetes | 1.135 |
| Indicator for oral antibiotic use | 1.128 |
| CCW indicator for anxiety disorders | 1.121 |
| Indicator for frailty | 1.120 |
| Indicator for respiratory infection | 1.119 |
| Indicator for albuminuria | 1.119 |
| CCW indicator for atrial fibrillation | 1.113 |
| Indicator for no vaccination (flu or pneumonia) | 1.108 |
| Indicator for diabetes with complications | 1.107 |
| CCW indicator for peripheral vascular disease | 1.093 |
| Number of emergency department visits within the past 6 | 1.093 |
| months | |
| Indicator for pneumonia | 1.089 |
| CCW indicator for depression and depressive disorders | 1.088 |
| Indicator for no anti-diabetes medication use | 1.083 |
| CCW indicator for Alzheimer's disease and related disorders | 1.082 |
| or senile dementia | |
| Indicator for provider administered drug | 1.081 |
| Located in whole county mental health care shortage area | 1.075 |
| Indicator for oral corticosteroid use | 1.071 |
| Beneficiary gender-female (relative to male) | 1.068 |
| Number of urgent care visits | 1.044 |
| Age | 1.022 |
| Percent with less than high school education, ages 65+ | 1.004 |
| Number of medications | 1.003 |
| Percent married | 0.995 |
| Number of prior admissions | 0.955 |
| Indicator for no beta blocker use | 0.952 |
| CCW indicator for rheumatoid arthritis/osteoarthritis | 0.943 |
| CCW indicator for glaucoma | 0.929 |
| Indicator for prior surgery | 0.905 |
| CCW indicator for cataracts | 0.905 |
| CCW indicator for hyperlipidemia | 0.895 |
| Air pollution level | 0.880 |
| CCW indicator for viral hepatitis | 0.875 |
| CCW indicator for personality disorders | 0.871 |
| Indicator for prior nursing home stay | 0.808 |
| Beneficiary race-Unknown | 0.772 |

It is important to note that risk factor coefficient estimates can change as the model is re-trained.

The outcome of the model is a set of probabilities that estimate the patient-specific risk of incurring an avoidable hospital event in the following months. In general, these events are rare: the MDPCP patient population numbers approximately 350,000 and there are typically 2,100 to 2,500 patients that experience at least one avoidable hospital event in a given month. As a consequence, the predicted probabilities are low. For patients with scores released in February 2020, the average probability of incurring an avoidable hospital event in the following month was 0.0066. Only 0.61 percent of the patient population had risk scores above 5 percent, and only forty-eight patients had risk scores above 50 percent. The system does not interpret this as a limitation of the risk scores; rather, this reflects the relative rarity of avoidable hospital events. Moreover, the relative risk is the key metric that should be used to allocate care resources: no matter the absolute risk of the patient panel, the efficient allocation of care resources requires the identification (and treatment) of the riskiest patients.

Patient-level risk tends to persist across time: that is, high-risk patients tend to remain high-risk from one month to the next, and low-risk patients tend to remain low-risk. For example, risk scores from May 2020 to June 2020 display a correlation of 0.98; from April 2020 to May 2020, the correlation is .97. This is likely due to two factors. First, in order to prevent coding idiosyncrasies from introducing noise into the predictions, all risk factors are coded with at least one year of lookback. This has the consequence of making the model risk factors relatively stable over time, and thus, smoothing out variation in the risk scores. Second, it is likely that true, underlying patient risk is also persistent: if some patients tend to have high (or low) risk for structural reasons, then the risk scores should also be relatively stable across time.

However, large month-to-month changes can occur in risk scores for two reasons. First, using a given set of risk factors coefficients, any changes in underlying risk factors will lead to changes in patients' predicted risk. For example, if an attributed beneficiary meets the conditions for heart failure beginning in July 2019, then her risk score will risk significantly from June 2019 to July 2019 because of that underlying change. Second, the system can estimate new risk factor coefficients every quarter (in the model re-training step). As a result, not only can the underlying risk factors for a given patient change from one month to the next, the relationship between that risk factor and the risk of avoidable hospital events can also change upon retraining. To continue the previous example, suppose that the model is retrained in July 2019, and that the risk factor coefficient for heart failure rises. As a consequence, that individual's risk score will now rise for two reasons: not only does she now have a heart failure risk factor, the heart failure risk factor has risen in predictive importance.

It is imperative that the accuracy of predictive models be assessed during both model development using validation data, and in a production environment. In the following, the results of both are presented.

Traditionally, the discriminatory power of predictive models has been summarized using the c-statistic, which is a measure of the area under the Receiver Operating Characteristic (ROC) curve. The ROC curve plots the true positive rate against the false positive rate for binary classifiers using successive cutoff thresholds and “measures the probability that a randomly selected diseased subject has a higher predicted risk than a randomly selected non-diseased subject”. However, this measure is uninformative regarding model calibration, which is the degree to which estimated risk scores match underlying true risk: it is possible to have a model with good discrimination and poor calibration. Moreover, the objective of the system is not binary classification, but instead the estimation of individual-level risks of incurring an avoidable hospital event so that care managers can, by focusing on the riskiest individuals, potentially intervene to prevent the most likely avoidable hospitalizations. To that end, the performance of the model is assessed using the concentration curve.

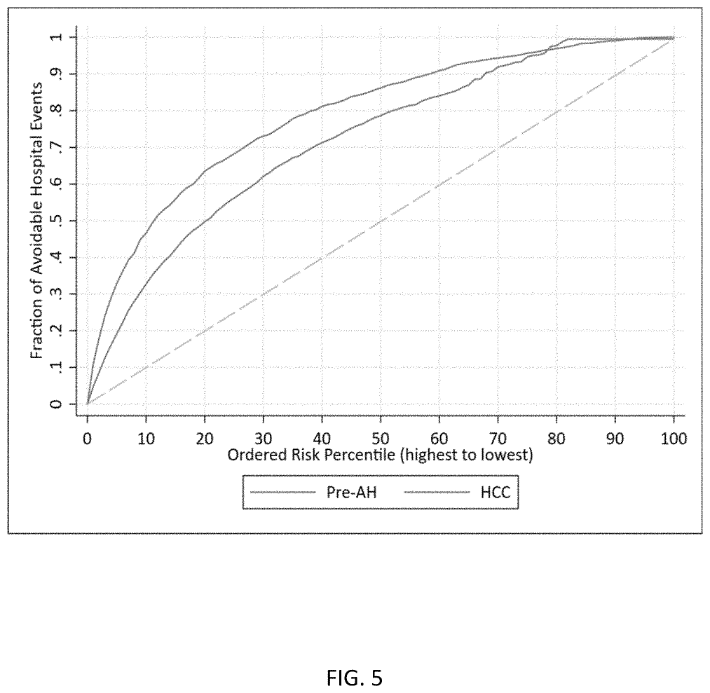

This measure of model accuracy estimates the cumulative share of all avoidable hospital events incurred by the riskiest patients, where the reader can determine the share of all avoidable hospital events occurring for individuals above different risk thresholds. In order to estimate the concentration curve, the patient cohort is ordered from most to least risky (in terms of predicted risk) on the X axis, and the fraction of total avoidable hospital events captured by the riskiest patients on the Y axis. In FIG. 5, the model scores released on Jan. 10, 2020, to estimate the concentration curve for these scores on actual avoidable hospital events for the period Jan. 10, 2020-Feb. 13, 2020. FIG. 5 is a graph showing concentration curves as of January 2020. It was found that the top 10 percent riskiest patients accounts for approximately 47 percent of all avoidable hospital events in the following month, and the top 20 percent riskiest patients account for almost two-thirds of all avoidable hospitalizations.

The same exercise were performed using the CMS HCC risk score for the January 2020 MDPCP attributed beneficiary cohort instead of the model risk score. This is also presented in FIG. 5. Note that the top 10 percent riskiest patients account for approximately 33 percent of all avoidable hospitalizations in the following month, and the top 20 percent riskiest patients account for approximately 50 percent of all avoidable hospitalizations. Given a baseline of approximately 2,500 avoidable hospital events per month, this implies that if care managers were to rely solely on the CMS HCC risk score and focus on the riskiest 10 percent of the cohort, then they would fail to identify 350 avoidable hospital events (relative to the number that would be identified using the model risk scores).

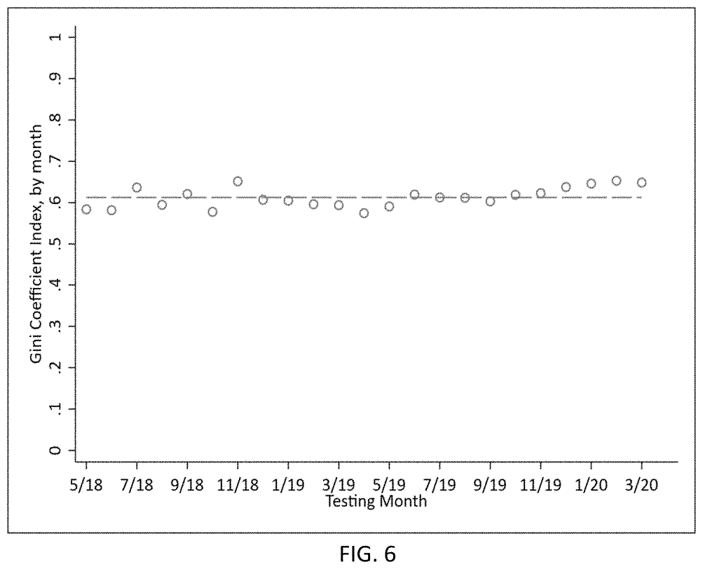

The model fit can also be summarized using the Gini coefficient, which is a measure (from 0 to 1) of the area between the concentration curve and the dotted 45-degree line. A higher Gini coefficient indicates better model fit. In order to assess whether model performance is improving or declining over time, we estimated monthly concentration curves for the 20 percent holdout sample of the training data set. While this is not production data, as above, this holdout data was not used to train the model, and therefore represents a test of model performance as if it were in a production environment. FIG. 6 is a graph that plots the Gini coefficients from 23 months of data based on the concentration curves estimated on the 20 percent holdout sample from each month. The final month of holdout data was not used due to incomplete information on avoidable hospital events in that month. These scores indicate that model performance is steady over time, with a very slight upward trend. This is consistent with the risk factors becoming more predictive as additional claims history becomes available.

Post-deployment model evaluation can be an important component of the predictive model lifecycle. The first model risk scores were released to participating providers on Oct. 11, 2019, and have been subsequently updated on the second Friday of every month since then. As of June 2020, the CCLF data appear to be reasonably complete through mid-April 2020, thus allowing for testing of the accuracy of the model predictions in a production environment for the first six months of scores.

In Table 9, below, these results are presented. For the scores released on Oct. 11, 2019, it was found that the top 10 percent riskiest patients accounted for 47.2 percent of all avoidable hospital events over the following month (until the next score release). Similar results were found for all other months of scores, with performance ranging from 45.0-47.3 percent.

TABLE 9
Predictive Performance of Pre-AH Model ™ Scores by Month

| Evaluation Period | Top 10% of Patients, by Risk |
|---|---|
| Oct. 11, 2019-Nov. 7, 2019 | 47.2% |
| Nov. 8, 2019-Dec. 12, 2019 | 47.3% |
| Dec. 13, 2019-Jan. 9, 2020 | 45.0% |
| Jan. 10, 2020-Feb. 13, 2020 | 46.6% |
| Feb. 14, 2020-Mar. 12, 2020 | 45.0% |
| Mar. 13, 2020-Apr. 9, 2020 | 45.9% |

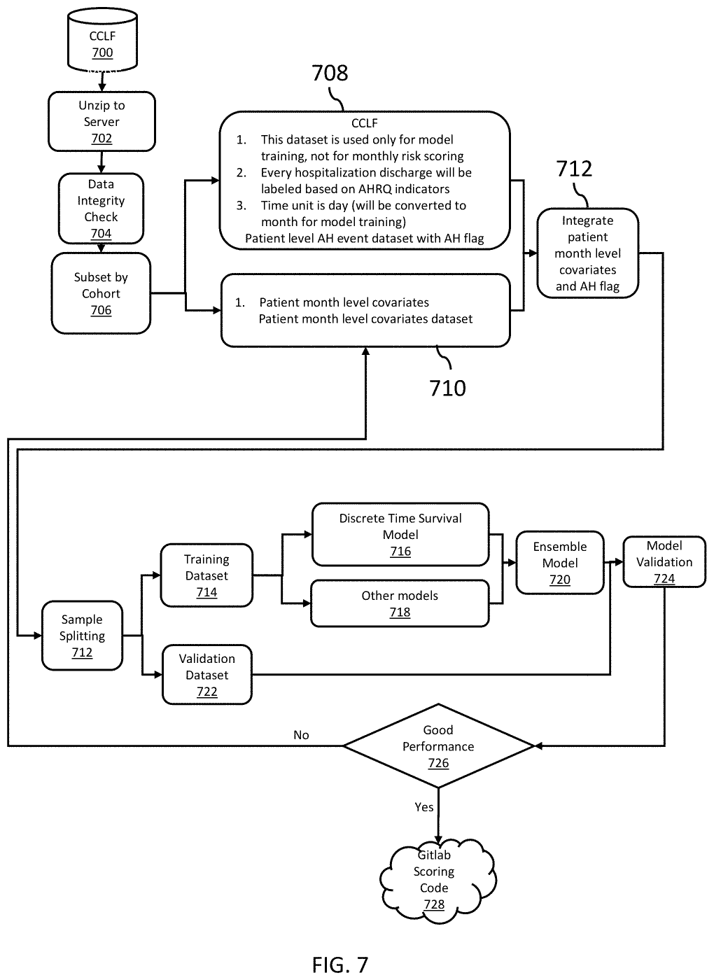

FIG. 7 illustrates a flow diagram of an example method for determining indicators of risk of patients to avoidable healthcare events in accordance with embodiments of the present disclosure. Referring to FIG. 7, initially a healthcare claims database 700 can be accessed, healthcare data of patients retrieved, and unzipped to a server (step 702). Subsequently, a data integrity check can be performed (step 704). At step 706, unique cohorts of individuals can be subset out of the claims bolus if needed. At block 708, the adverse events of interest are created for the model training analytical dataset at the discrete time level. At block 710, the risk factor covariates are created for the model training analytical dataset at the discrete time level. At step 712, patient month level covariates and event flags are integrated.

At step 712, sample splitting is conducted by the system and a training dataset generated (step 714). At step 716, a discrete time survival model is applied to the training dataset 714. Concurrently, step 718 applies any competing models to be tested during the current model training cycle. At step 720, the results of the model 716 and any model(s) 718 are combined in an ensamble to create a single risk scoring algorithm. Step 724 applies the scoring algorithm 720 to the validation datset 722 to test model performance. At block 726, the system determines whether the performance from 724 is acceptable for deployment. If it is determined that there is not good performance, the method returns to step 712. Otherwise, if it is determined that there is good performance, the method generates a scoring code (step 728).

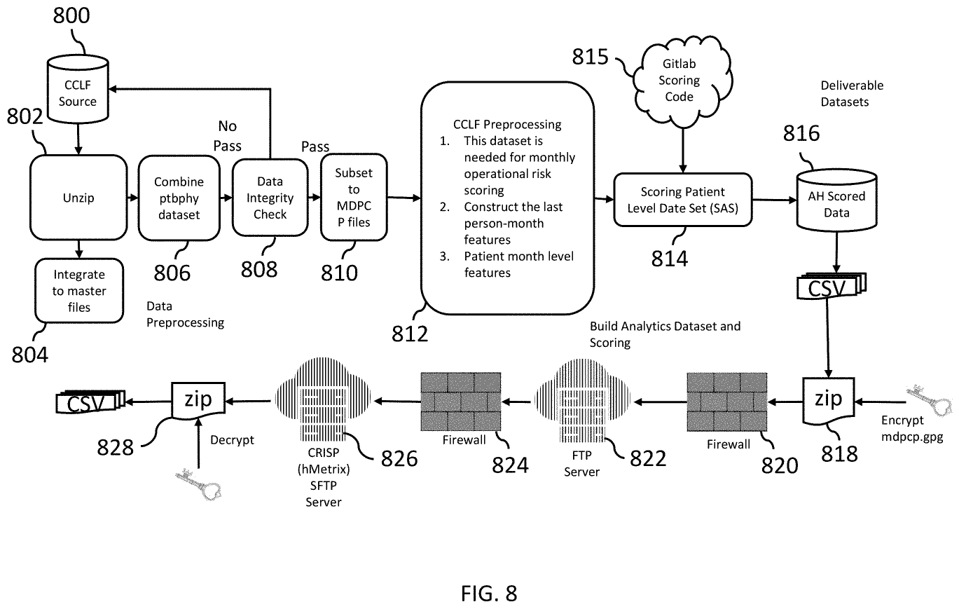

FIG. 8 illustrates a flow diagram of an example method for scoring and file transfer processing in accordance with embodiments of the present disclosure. Referring to FIG. 8, the method includes providing a healthcare claims source 800. The system can unzip and access specified data from the source 800 at step 802. At step 804, new healthcare claims are added to a historical database that contains all claims received during each subsequent data loads. At step 806, any claims files that were split apart due to data transfer constrains are merged back together. At step 808, data integrity is checked.

Step 812 includes claiming data preprocessing. All events and covariates are defined and the global analytical dataset is generated at the person-month level. At step 814, the most recent temporal record for each individual is pulled out and scored using scoring code 815. At step 816, the scored cohort file is created is prepared for export. At step 818, the scored export file is compressed and encrypted. A firewall is provided subsequently at step 820. At step 822, the scored export file is passed to a secure FTP server. A firewall is provided subsequently at step 824. At step 826, the scored export file is passed to another secure FTP server. At step 828, the scored export file is incorporated into the health information exchange.

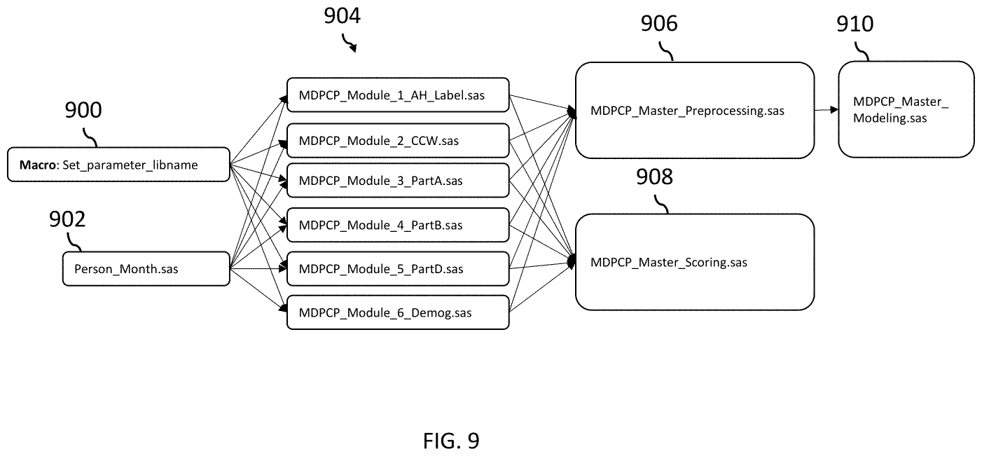

FIG. 9 illustrates a diagram showing code modularity according to embodiments of the present disclosure. Referring to FIG. 9, six modules are shared by preprocessing and scoring processes. The goal of including Person_Month.sas and macro in each module is to make it independently executable. At steps 900 and 902, global macro coding libraries are defined and available person-month combinations are identified for analysis. At the blocks designed by reference 904, create each of the adverse event and risk factor covaries for each identified person-month. At step 908, scoring code is applied to the most recent available person-month, resulting in risk scores and reasons for risk. At steps 906 and 910, the model is re-trained based on the most recent available data, and updated scoring code is archived for the next iteration.

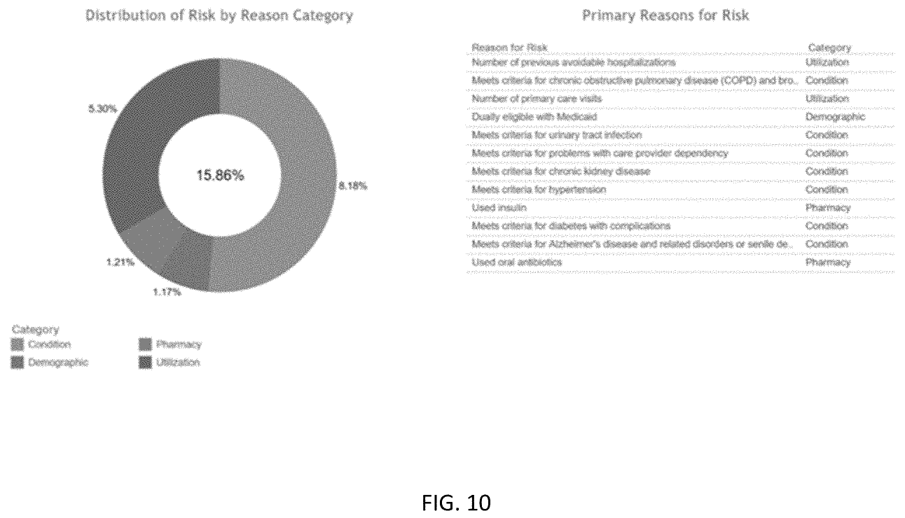

FIG. 10 is a screenshot of a display of a patient's data including likelihood of avoidable hospital event in accordance with embodiments of the present disclosure. Referring to FIG. 10, the display indicates that this patient has a 15.86% risk for an avoidable hospital event in general. Also, the display indicates other specific risks for the patient by category.

The functional units described in this specification have been labeled as computing devices. A computing device may be implemented in programmable hardware devices such as processors, digital signal processors, central processing units, field programmable gate arrays, programmable array logic, programmable logic devices, cloud processing systems, or the like. The computing devices may also be implemented in software for execution by various types of processors. An identified device may include executable code and may, for instance, comprise one or more physical or logical blocks of computer instructions, which may, for instance, be organized as an object, procedure, function, or other construct. Nevertheless, the executable of an identified device need not be physically located together but may comprise disparate instructions stored in different locations which, when joined logically together, comprise the computing device and achieve the stated purpose of the computing device. In another example, a computing device may be a server or other computer located within a retail environment and communicatively connected to other computing devices (e.g., POS equipment or computers) for managing accounting, purchase transactions, and other processes within the retail environment. In another example, a computing device may be a mobile computing device such as, for example, but not limited to, a smartphone, a cell phone, a pager, a personal digital assistant (PDA), a mobile computer with a smartphone client, or the like. In another example, a computing device may be any type of wearable computer, such as a computer with a head-mounted display (HIVID), or a smart watch or some other wearable smart device. Some of the computer sensing may be part of the fabric of the clothes the user is wearing. A computing device can also include any type of conventional computer, for example, a laptop computer or a tablet computer. A typical mobile computing device is a wireless data access-enabled device (e.g., an iPHONE® smartphone, a SAMSUNG® smartphone, an iPAD® device, smart watch, or the like) that is capable of sending and receiving data in a wireless manner using protocols like the Internet Protocol, or IP, and the wireless application protocol, or WAP. This allows users to access information via wireless devices, such as smart watches, smartphones, mobile phones, pagers, two-way radios, communicators, and the like. Wireless data access is supported by many wireless networks, including, but not limited to, Bluetooth, Near Field Communication, CDPD, CDMA, GSM, PDC, PHS, TDMA, FLEX, ReFLEX, iDEN, TETRA, DECT, DataTAC, Mobitex, EDGE and other 2G, 3G, 4G, 5G, and LTE technologies, and it operates with many handheld device operating systems, such as PalmOS, EPOC, Windows CE, FLEXOS, OS/9, JavaOS, iOS and Android. Typically, these devices use graphical displays and can access the Internet (or other communications network) on so-called mini- or micro-browsers, which are web browsers with small file sizes that can accommodate the reduced memory constraints of wireless networks. In a representative embodiment, the mobile device is a cellular telephone or smartphone or smart watch that operates over GPRS (General Packet Radio Services), which is a data technology for GSM networks or operates over Near Field Communication e.g. Bluetooth. In addition to a conventional voice communication, a given mobile device can communicate with another such device via many different types of message transfer techniques, including Bluetooth, Near Field Communication, SMS (short message service), enhanced SMS (EMS), multi-media message (MMS), email WAP, paging, or other known or later-developed wireless data formats. Although many of the examples provided herein are implemented on smartphones, the examples may similarly be implemented on any suitable computing device, such as a computer.

An executable code of a computing device may be a single instruction, or many instructions, and may even be distributed over several different code segments, among different applications, and across several memory devices. Similarly, operational data may be identified and illustrated herein within the computing device, and may be embodied in any suitable form and organized within any suitable type of data structure. The operational data may be collected as a single data set, or may be distributed over different locations including over different storage devices, and may exist, at least partially, as electronic signals on a system or network.

The described features, structures, or characteristics may be combined in any suitable manner in one or more embodiments. In the following description, numerous specific details are provided, to provide a thorough understanding of embodiments of the disclosed subject matter. One skilled in the relevant art will recognize, however, that the disclosed subject matter can be practiced without one or more of the specific details, or with other methods, components, materials, etc. In other instances, well-known structures, materials, or operations are not shown or described in detail to avoid obscuring aspects of the disclosed subject matter.

The device or system for performing one or more operations on a memory of a computing device may be a software, hardware, firmware, or combination of these. The device or the system is further intended to include or otherwise cover all software or computer programs capable of performing the various heretofore-disclosed determinations, calculations, or the like for the disclosed purposes. For example, exemplary embodiments are intended to cover all software or computer programs capable of enabling processors to implement the disclosed processes. Exemplary embodiments are also intended to cover any and all currently known, related art or later developed non-transitory recording or storage mediums (such as a CD-ROM, DVD-ROM, hard drive, RAM, ROM, floppy disc, magnetic tape cassette, etc.) that record or store such software or computer programs. Exemplary embodiments are further intended to cover such software, computer programs, systems and/or processes provided through any other currently known, related art, or later developed medium (such as transitory mediums, carrier waves, etc.), usable for implementing the exemplary operations disclosed below.

In accordance with the exemplary embodiments, the disclosed computer programs can be executed in many exemplary ways, such as an application that is resident in the memory of a device or as a hosted application that is being executed on a server and communicating with the device application or browser via a number of standard protocols, such as TCP/IP, HTTP, XML, SOAP, REST, JSON and other sufficient protocols. The disclosed computer programs can be written in exemplary programming languages that execute from memory on the device or from a hosted server, such as BASIC, COBOL, C, C++, Java, Pascal, or scripting languages such as JavaScript, Python, Ruby, PHP, Perl, or other suitable programming languages.

The present subject matter may be a system, a method, and/or a computer program product. The computer program product may include a computer readable storage medium (or media) having computer readable program instructions thereon for causing a processor to carry out aspects of the present subject matter.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a RAM, a ROM, an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network, or Near Field Communication. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present subject matter may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Java, Smalltalk, C++, Javascript or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present subject matter.

Aspects of the present subject matter are described herein with reference to flowchart illustrations and/or block diagrams of methods, apparatus (systems), and computer program products according to embodiments of the subject matter. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present subject matter. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

While the embodiments have been described in connection with the various embodiments of the various figures, it is to be understood that other similar embodiments may be used, or modifications and additions may be made to the described embodiment for performing the same function without deviating therefrom. Therefore, the disclosed embodiments should not be limited to any single embodiment, but rather should be construed in breadth and scope in accordance with the appended claims.

### APPENDIX 1: RISK FACTOR CODEBOOK

In the following, information of a risk factor codebook in accordance with embodiments of the present disclosure is provided.

Age: For each person-month, this variable records person age as of the end of the month.

### Source: Beneficiary Demographics

| Risk Factor | Mean | Minimum | Maximum |
|---|---|---|---|
| Age | 72.429 | 19 | 108 |

Number of avoidable hospitalizations: For each person-month, this variable counts the number of avoidable hospitalizations incurred within the prior 12 months (not including the month in which the avoidable hospitalization occurred).
Source: Part A claims

| Risk Factor | Mean | Minimum | Maximum |
|---|---|---|---|
| Number of avoidable hospitalizations | .058 | 0 | 49 |

Indicator for no anti-diabetes medication use: For each person-month, this variable takes the value of 1 if a person did not incur a claim for anti-diabetes medication within the past 12 months, and 0 otherwise.
Source: Part D claims

| Risk Factor | Mean | Minimum | Maximum |
|---|---|---|---|
| Indicator for no anti-diabetes | .965 | 0 | 1 |
| medication use | | | |

Indicator for no beta blocker use: For each person-month, this variable takes the value of 1 if a person did not incur a claim for beta blockers within the past 12 months, and 0 otherwise.
Source: Part D claims

| Risk Factor | Mean | Minimum | Maximum |
|---|---|---|---|
| Indicator for no beta blocker use | .832 | 0 | 1 |

CCW indicator for acquired hypothyroidism: For each person-month, this variable records whether the person meets the CCW clinical criteria for acquired hypothyroidism. If so, this variable takes the value 1; if not, then 0.
Source: Part A and B claims

| Risk Factor | Mean | Minimum | Maximum |
|---|---|---|---|
| CCW indicator for acquired | .135 | 0 | 1 |
| hypothyroidism | | | |

CCW indicator for acute myocardial infarction: For each person-month, this variable records whether the person meets the CCW clinical criteria for acute myocardial infarction. If so, this variable takes the value 1; if not, then 0.
Source: Part A and B claims

| Risk Factor | Mean | Minimum | Maximum |
|---|---|---|---|
| CCW indicator for acute | .005 | 0 | 1 |
| myocardial infarction | | | |

CCW indicator for ADHD, conduct disorders, and hyperkinetic syndrome: For each person-month, this variable records whether the person meets the CCW clinical criteria for ADHD, conduct disorders, and hyperkinetic syndrome. If so, this variable takes the value 1; if not, then 0.
Source: Part A and B claims

| Risk Factor | Mean | Minimum | Maximum |
|---|---|---|---|
| CCW indicator for ADHD, conduct | .006 | 0 | 1 |
| disorders, and hyperkinetic syndrome | | | |

Indicator for albuminuria: For each person-month, this variable records whether the person has incurred at least one inpatient or two non-inpatient claims with any diagnosis for albuminuria within the past two years. If so, this variable takes the value 1; if not, then 0.
Source: Part A and B claims

| Risk Factor | Mean | Minimum | Maximum |
|---|---|---|---|
| Indicator for albuminuria | .015 | 0 | 1 |

CCW indicator for alcohol use disorders: For each person-month, this variable records whether the person meets the CCW clinical criteria for alcohol use disorders. If so, this variable takes the value 1; if not, then 0.
Source: Part A and B claims

| Risk Factor | Mean | Minimum | Maximum |
|---|---|---|---|
| CCW indicator for alcohol use disorders | .002 | 0 | 1 |

CCW indicator for Alzheimer's disease and related disorders or senile dementia: For each person-month, this variable records whether the person meets the CCW clinical criteria for Alzheimer's disease and related disorders or senile dementia. If so, this variable takes the value 1; if not, then 0.
Source: Part A and B claims

| Risk Factor | Mean | Minimum | Maximum |
|---|---|---|---|
| CCW indicator of Alzheimer’s disease | .051 | 0 | 1 |
| and related disorders or senile dementia | | | |

CCW indicator for anemia: For each person-month, this variable records whether the person meets the CCW clinical criteria for anemia. If so, this variable takes the value 1; if not, then 0.
Source: Part A and B claims

| Risk Factor | Mean | Minimum | Maximum |
|---|---|---|---|
| CCW indicator for anemia | .182 | 0 | 1 |

CCW indicator for anxiety disorders: For each person-month, this variable records whether the person meets the CCW clinical criteria for anxiety disorders. If so, this variable takes the value 1; if not, then 0.
Source: Part A and B claims

| Risk Factor | Mean | Minimum | Maximum |
|---|---|---|---|
| CCW indicator for anxiety disorders | .112 | 0 | 1 |

Indicator for arrhythmia: For each person-month, this variable records whether the person has incurred at least one inpatient or two non-inpatient claims with any diagnosis for arrhythmia within the past two years. If so, this variable takes the value 1; if not, then 0.
Source: Part A and B claims

| Risk Factor | Mean | Minimum | Maximum |
|---|---|---|---|
| Indicator for arrhythmia | .16 | 0 | 1 |

CCW indicator for asthma: For each person-month, this variable records whether the person meets the CCW clinical criteria for asthma. If so, this variable takes the value 1; if not, then 0.
Source: Part A and B claims

| Risk Factor | Mean | Minimum | Maximum |
|---|---|---|---|
| CCW indicator for asthma | .048 | 0 | 1 |

CCW indicator for atrial fibrillation: For each person-month, this variable records whether the person meets the CCW clinical criteria for atrial fibrillation. If so, this variable takes the value 1; if not, then 0.
Source: Part A and B claims

| Risk Factor | Mean | Minimum | Maximum |
|---|---|---|---|
| CCW indicator for | .065 | 0 | 1 |
| atrial fibrillation | | | |

CCW indicator for autism spectrum disorders: For each person-month, this variable records whether the person meets the CCW clinical criteria for autism spectrum disorders. If so, this variable takes the value 1; if not, then 0.
Source: Part A and B claims

| Risk Factor | Mean | Minimum | Maximum |
|---|---|---|---|
| CCW indicator for autism | .002 | 0 | 1 |
| spectrum disorders | | | |

CCW indicator for benign prostatic hyperplasia: For each person-month, this variable records whether the person meets the CCW clinical criteria for benign prostatic hyperplasia. If so, this variable takes the value 1; if not, then 0.
Source: Part A and B claims

| Risk Factor | Mean | Minimum | Maximum |
|---|---|---|---|
| CCW indicator for benign | .066 | 0 | 1 |
| prostatic hyperplasia | | | |

CCW indicator for bipolar disorder: For each person-month, this variable records whether the person meets the CCW clinical criteria for bipolar disorder. If so, this variable takes the value 1; if not, then 0.
Source: Part A and B claims

| Risk Factor | Mean | Minimum | Maximum |
|---|---|---|---|
| CCW indicator for | .02 | 0 | 1 |
| bipolar disorder | | | |

CCW indicator for cataracts: For each person-month, this variable records whether the person meets the CCW clinical criteria for cataracts. If so, this variable takes the value 1; if not, then 0.
Source: Part A and B claims

| Risk Factor | Mean | Minimum | Maximum |
|---|---|---|---|
| CCW indicator for cataracts | .166 | 0 | 1 |

CCW indicator for cerebral palsy: For each person-month, this variable records whether the person meets the CCW clinical criteria for cerebral palsy. If so, this variable takes the value 1; if not, then 0.
Source: Part A and B claims

| Risk Factor | Mean | Minimum | Maximum |
|---|---|---|---|
| CCW indicator for cerebral palsy | .002 | 0 | 1 |

Indicator for cerebrovascular disease: For each person-month, this variable records whether the person has incurred at least one inpatient or two non-inpatient claims with any diagnosis for cerebrovascular disease within the past two years. If so, this variable takes the value 1; if not, then 0.
Source: Part A and B claims

| Risk Factor | Mean | Minimum | Maximum |
|---|---|---|---|
| Indicator for cerebrovascular disease | .09 | 0 | 1 |

CCW indicator for chronic kidney disease: For each person-month, this variable records whether the person meets the CCW clinical criteria for chronic kidney disease. If so, this variable takes the value 1; if not, then 0.
Source: Part A and B claims

| Risk Factor | Mean | Minimum | Maximum |
|---|---|---|---|
| CCW indicator for chronic | .182 | 0 | 1 |
| kidney disease | | | |

CCW indicator for chronic obstructive pulmonary disease (COPD) and bronchiectasis: For each person-month, this variable records whether the person meets the CCW clinical criteria for chronic obstructive pulmonary disease (COPD) and bronchiectasis. If so, this variable takes the value 1; if not, then 0.
Source: Part A and B claims

| Risk Factor | Mean | Minimum | Maximum |
|---|---|---|---|
| CCW indicator for chronic obstructive | .077 | 0 | 1 |
| pulmonary disease (COPD) and | | | |
| bronchiectasis | | | |

CCW indicator for colorectal cancer: For each person-month, this variable records whether the person meets the CCW clinical criteria for colorectal cancer. If so, this variable takes the value 1; if not, then 0.
Source: Part A and B claims

| Risk Factor | Mean | Minimum | Maximum |
|---|---|---|---|
| CCW indicator for | .009 | 0 | 1 |
| colorectal cancer | | | |

CCW indicator for cystic fibrosis and other metabolic developmental disorders: For each person-month, this variable records whether the person meets the CCW clinical criteria for cystic fibrosis and other metabolic developmental disorders. If so, this variable takes the value 1; if not, then 0.
Source: Part A and B claims

| Risk Factor | Mean | Minimum | Maximum |
|---|---|---|---|
| CCW indicator for cystic fibrosis and | .004 | 0 | 1 |
| other metabolic developmental disorders | | | |

CCW indicator for depression and depressive disorders: For each person-month, this variable records whether the person meets the CCW clinical criteria for depression and depressive disorders. If so, this variable takes the value 1; if not, then 0.
Source: Part A and B claims

| Risk Factor | Mean | Minimum | Maximum |
|---|---|---|---|
| CCW indicator for depression | .122 | 0 | 1 |
| and depressive disorders | | | |

CCW indicator for diabetes: For each person-month, this variable records whether the person meets the CCW clinical criteria for diabetes. If so, this variable takes the value 1; if not, then 0.
Source: Part A and B claims

| Risk Factor | Mean | Minimum | Maximum |
|---|---|---|---|
| CCW indicator for diabetes | .251 | 0 | 1 |

Indicator for diabetes with complications: For each person-month, this variable records whether the person has incurred at least one inpatient or two non-inpatient claims with any diagnosis for diabetes with complications within the past two years. If so, this variable takes the value 1; if not, then 0.
Source: Part A and B claims

| Risk Factor | Mean | Minimum | Maximum |
|---|---|---|---|
| Indicator for diabetes | .148 | 0 | 1 |
| with complications | | | |

Indicator for diabetic ulcer: For each person-month, this variable records whether the person has incurred at least one inpatient or two non-inpatient claims with any diagnosis for diabetic ulcer within the past two years. If so, this variable takes the value 1; if not, then 0.
Source: Part A and B claims

| Risk Factor | Mean | Minimum | Maximum |
|---|---|---|---|
| Indicator for diabetic ulcer | .032 | 0 | 1 |

CCW indicator for drug use disorders: For each person-month, this variable records whether the person meets the CCW clinical criteria for drug use disorders. If so, this variable takes the value 1; if not, then 0.
Source: Part A and B claims

| Risk Factor | Mean | Minimum | Maximum |
|---|---|---|---|
| CCW indicator for drug | .002 | 0 | 1 |
| use disorders | | | |

CCW indicator for endometrial cancer: For each person-month, this variable records whether the person meets the CCW clinical criteria for endometrial cancer. If so, this variable takes the value 1; if not, then 0.
Source: Part A and B claims

| Risk Factor | Mean | Minimum | Maximum |
|---|---|---|---|
| CCW indicator for | .004 | 0 | 1 |
| endometrial cancer | | | |

CCW indicator for epilepsy: For each person-month, this variable records whether the person meets the CCW clinical criteria for epilepsy. If so, this variable takes the value 1; if not, then 0.
Source: Part A and B claims

| Risk Factor | Mean | Minimum | Maximum |
|---|---|---|---|
| CCW indicator for epilepsy | .016 | 0 | 1 |

CCW indicator for female/male breast cancer: For each person-month, this variable records whether the person meets the CCW clinical criteria for female/male breast cancer. If so, this variable takes the value 1; if not, then 0.
Source: Part A and B claims

| Risk Factor | Mean | Minimum | Maximum |
|---|---|---|---|
| CCW indicator for female/ | .035 | 0 | 1 |
| male breast cancer | | | |

CCW indicator for fibromyalgia, chronic pain and fatigue: For each person-month, this variable records whether the person meets the CCW clinical criteria for fibromyalgia, chronic pain and fatigue. If so, this variable takes the value 1; if not, then 0.
Source: Part A and B claims

| Risk Factor | Mean | Minimum | Maximum |
|---|---|---|---|
| CCW indicator for fibromyalgia, | .154 | 0 | 1 |
| chronic pain and fatigue | | | |

Indicator for fluid and electrolyte imbalance: For each person-month, this variable records whether the person has incurred at least one inpatient or two non-inpatient claims with any diagnosis for fluid and electrolyte imbalance within the past two years. If so, this variable takes the value 1; if not, then 0.
Source: Part A and B claims

| Risk Factor | Mean | Minimum | Maximum |
|---|---|---|---|
| Indicator for fluid and | .095 | 0 | 1 |
| electrolyte imbalance | | | |

Indicator for gastroesophageal reflux disease: For each person-month, this variable records whether the person has incurred at least one inpatient or two non-inpatient claims with any diagnosis for gastroesophageal reflux disease within the past two years. If so, this variable takes the value 1; if not, then 0.
Source: Part A and B claims

| Risk Factor | Mean | Minimum | Maximum |
|---|---|---|---|
| Indicator for gastroesophageal | .171 | 0 | 1 |
| reflux disease | | | |

Indicator for gastroparesis: For each person-month, this variable records whether the person has incurred at least one inpatient or two non-inpatient claims with any diagnosis for gastroparesis within the past two years. If so, this variable takes the value 1; if not, then 0.
Source: Part A and B claims

| Risk Factor | Mean | Minimum | Maximum |
|---|---|---|---|
| Indicator for gastroparesis | .003 | 0 | 1 |

CCW indicator for glaucoma: For each person-month, this variable records whether the person meets the CCW clinical criteria for glaucoma. If so, this variable takes the value 1; if not, then 0.
Source: Part A and B claims

| Risk Factor | Mean | Minimum | Maximum |
|---|---|---|---|
| CCW indicator for glaucoma | .12 | 0 | 1 |

CCW indicator for heart failure: For each person-month, this variable records whether the person meets the CCW clinical criteria for heart failure. If so, this variable takes the value 1; if not, then 0.
Source: Part A and B claims

| Risk Factor | Mean | Minimum | Maximum |
|---|---|---|---|
| CCW indicator for heart failure | .076 | 0 | 1 |

CCW indicator for hip/pelvic fracture: For each person-month, this variable records whether the person meets the CCW clinical criteria for hip/pelvic fracture. If so, this variable takes the value 1; if not, then 0.
Source: Part A and B claims

| Risk Factor | Mean | Minimum | Maximum |
|---|---|---|---|
| CCW indicator for hip/pelvic fracture | .003 | 0 | 1 |

CCW indicator for HIV/AIDS: For each person-month, this variable records whether the person meets the CCW clinical criteria for HIV/AIDS. If so, this variable takes the value 1; if not, then 0.
Source: Part A and B claims

| Risk Factor | Mean | Minimum | Maximum |
|---|---|---|---|
| CCW indicator for HIV/AIDS | .003 | 0 | 1 |

CCW indicator for hyperlipidemia: For each person-month, this variable records whether the person meets the CCW clinical criteria for hyperlipidemia. If so, this variable takes the value 1; if not, then 0.
Source: Part A and B claims

| Risk Factor | Mean | Minimum | Maximum |
|---|---|---|---|
| CCW indicator for hyperlipidemia | .498 | 0 | 1 |

CCW indicator for hypertension: For each person-month, this variable records whether the person meets the CCW clinical criteria for hypertension. If so, this variable takes the value 1; if not, then 0.
Source: Part A and B claims

| Risk Factor | Mean | Minimum | Maximum |
|---|---|---|---|
| CCW indicator for hypertension | .565 | 0 | 1 |

CCW indicator for intellectual disabilities and related conditions: For each person-month, this variable records whether the person meets the CCW clinical criteria for intellectual disabilities and related conditions. If so, this variable takes the value 1; if not, then 0.
Source: Part A and B claims

| Risk Factor | Mean | Minimum | Maximum |
|---|---|---|---|
| CCW indicator for intellectual | .007 | 0 | 1 |
| disabilities and related conditions | | | |

CCW indicator for ischemic heart disease: For each person-month, this variable records whether the person meets the CCW clinical criteria for ischemic heart disease. If so, this variable takes the value 1; if not, then 0.
Source: Part A and B claims

| Risk Factor | Mean | Minimum | Maximum |
|---|---|---|---|
| CCW indicator for ischemic | .207 | 0 | 1 |
| heart disease | | | |

CCW indicator for learning disabilities: For each person-month, this variable records whether the person meets the CCW clinical criteria for learning disabilities. If so, this variable takes the value 1; if not, then 0.
Source: Part A and B claims

| Risk Factor | Mean | Minimum | Maximum |
|---|---|---|---|
| CCW indicator for learning disabilities | .001 | 0 | 1 |

CCW indicator for leukemias and lymphomas: For each person-month, this variable records whether the person meets the CCW clinical criteria for leukemias and lymphomas. If so, this variable takes the value 1; if not, then 0.
Source: Part A and B claims

| Risk Factor | Mean | Minimum | Maximum |
|---|---|---|---|
| CCW indicator for | .012 | 0 | 1 |
| leukemias and lymphomas | | | |

CCW indicator for liver disease, cirrhosis and other liver conditions (except viral hepatitis): For each person-month, this variable records whether the person meets the CCW clinical criteria for liver disease, cirrhosis and other liver conditions (except viral hepatitis). If so, this variable takes the value 1; if not, then 0.
Source: Part A and B claims

| Risk Factor | Mean | Minimum | Maximum |
|---|---|---|---|
| CCW indicator for liver disease, | .028 | 0 | 1 |
| cirrhosis and other liver | | | |
| conditions (except viral hepatitis) | | | |

CCW indicator for lung cancer: For each person-month, this variable records whether the person meets the CCW clinical criteria for lung cancer. If so, this variable takes the value 1; if not, then 0.
Source: Part A and B claims

| Risk Factor | Mean | Minimum | Maximum |
|---|---|---|---|
| CCW indicator for lung cancer | .008 | 0 | 1 |

Indicator for metastatic cancer: For each person-month, this variable records whether the person has incurred at least one inpatient or two non-inpatient claims with any diagnosis for metastatic cancer within the past two years. If so, this variable takes the value 1; if not, then 0.
Source: Part A and B claims

| Risk Factor | Mean | Minimum | Maximum |
|---|---|---|---|
| Indicator for metastatic cancer | .011 | 0 | 1 |

CCW indicator for migraine and chronic headache: For each person-month, this variable records whether the person meets the CCW clinical criteria for migraine and chronic headache. If so, this variable takes the value 1; if not, then 0.
Source: Part A and B claims

| Risk Factor | Mean | Minimum | Maximum |
|---|---|---|---|
| CCW indicator for migraine | .024 | 0 | 1 |
| and chronic headache | | | |

CCW indicator for mobility impairments: For each person-month, this variable records whether the person meets the CCW clinical criteria for mobility impairments. If so, this variable takes the value 1; if not, then 0.
Source: Part A and B claims

| Risk Factor | Mean | Minimum | Maximum |
|---|---|---|---|
| CCW indicator for mobility impairments | .015 | 0 | 1 |

CCW indicator for multiple sclerosis and transverse myelitis: For each person-month, this variable records whether the person meets the CCW clinical criteria for multiple sclerosis and transverse myelitis. If so, this variable takes the value 1; if not, then 0.
Source: Part A and B claims

| Risk Factor | Mean | Minimum | Maximum |
|---|---|---|---|
| CCW indicator for multiple | .005 | 0 | 1 |
| sclerosis and transverse myelitis | | | |

CCW indicator for muscular dystrophy: For each person-month, this variable records whether the person meets the CCW clinical criteria for muscular dystrophy. If so, this variable takes the value 1; if not, then 0.
Source: Part A and B claims

| Risk Factor | Mean | Minimum | Maximum |
|---|---|---|---|
| CCW indicator for muscular dystrophy | 0 | 0 | 1 |

Indicator for neuropathy: For each person-month, this variable records whether the person has incurred at least one inpatient or two non-inpatient claims with any diagnosis for neuropathy within the past two years. If so, this variable takes the value 1; if not, then 0.
Source: Part A and B claims

| Risk Factor | Mean | Minimum | Maximum |
|---|---|---|---|
| Indicator for neuropathy | .051 | 0 | 1 |

CCW indicator for obesity: For each person-month, this variable records whether the person meets the CCW clinical criteria for obesity. If so, this variable takes the value 1; if not, then 0.
Source: Part A and B claims

| Risk Factor | Mean | Minimum | Maximum |
|---|---|---|---|
| CCW indicator for obesity | .15 | 0 | 1 |

Indicator for occupational exposure to risk factors: For each person-month, this variable records whether the person has incurred at least one inpatient or two non-inpatient claims with any diagnosis for occupational exposure to risk factors within the past two years. If so, this variable takes the value 1; if not, then 0.
Source: Part A and B claims

| Risk Factor | Mean | Minimum | Maximum |
|---|---|---|---|
| Indicator for occupational exposure to | 0 | 0 | 1 |
| risk factors | | | |

CCW indicator for osteoporosis: For each person-month, this variable records whether the person meets the CCW clinical criteria for osteoporosis. If so, this variable takes the value 1; if not, then 0.
Source: Part A and B claims

| Risk Factor | Mean | Minimum | Maximum |
|---|---|---|---|
| CCW indicator for osteoporosis | .06 | 0 | 1 |

CCW indicator for other developmental delays: For each person-month, this variable records whether the person meets the CCW clinical criteria for other developmental delays. If so, this variable takes the value 1; if not, then 0.
Source: Part A and B claims

| Risk Factor | Mean | Minimum | Maximum |
|---|---|---|---|
| CCW indicator for other developmental | .001 | 0 | 1 |
| delays | | | |

Indicator for other problems with primary support group: For each person-month, this variable records whether the person has incurred at least one inpatient or two non-inpatient claims with any diagnosis for other problems with primary support group within the past two years. If so, this variable takes the value 1; if not, then 0.
Source: Part A and B claims

| Risk Factor | Mean | Minimum | Maximum |
|---|---|---|---|
| Indicator for other problems with primary | .002 | 0 | 1 |
| support group | | | |

Indicator for pancreatitis: For each person-month, this variable records whether the person has incurred at least one inpatient or two non-inpatient claims with any diagnosis for pancreatitis within the past two years. If so, this variable takes the value 1; if not, then 0.
Source: Part A and B claims

| Risk Factor | Mean | Minimum | Maximum |
|---|---|---|---|
| Indicator for pancreatitis | .009 | 0 | 1 |

Indicator for peptic ulcer disease: For each person-month, this variable records whether the person has incurred at least one inpatient or two non-inpatient claims with any diagnosis for peptic ulcer disease within the past two years. If so, this variable takes the value 1; if not, then 0.
Source: Part A and B claims

| Risk Factor | Mean | Minimum | Maximum |
|---|---|---|---|
| Indicator for peptic ulcer disease | .008 | 0 | 1 |

Indicator for peripheral and visceral atherosclerosis: For each person-month, this variable records whether the person has incurred at least one inpatient or two non-inpatient claims with any diagnosis for peripheral and visceral atherosclerosis within the past two years. If so, this variable takes the value 1; if not, then 0.
Source: Part A and B claims

| Risk Factor | Mean | Minimum | Maximum |
|---|---|---|---|
| Indicator for peripheral and visceral | .083 | 0 | 1 |
| atherosclerosis | | | |

CCW indicator for peripheral vascular disease: For each person-month, this variable records whether the person meets the CCW clinical criteria for peripheral vascular disease. If so, this variable takes the value 1; if not, then 0.
Source: Part A and B claims

| Risk Factor | Mean | Minimum | Maximum |
|---|---|---|---|
| CCW indicator for peripheral vascular | .097 | 0 | 1 |
| disease | | | |

CCW indicator for personality disorders: For each person-month, this variable records whether the person meets the CCW clinical criteria for personality disorders. If so, this variable takes the value 1; if not, then 0.
Source: Part A and B claims

| Risk Factor | Mean | Minimum | Maximum |
|---|---|---|---|
| CCW indicator for personality disorders | .009 | 0 | 1 |

Indicator for pneumonia: For each person-month, this variable records whether the person has incurred at least one inpatient or two non-inpatient claims with any diagnosis for pneumonia within the past two years. If so, this variable takes the value 1; if not, then 0.
Source: Part A and B claims

| Risk Factor | Mean | Minimum | Maximum |
|---|---|---|---|
| Indicator for pneumonia | .033 | 0 | 1 |

CCW indicator for post-traumatic stress disorder: For each person-month, this variable records whether the person meets the CCW clinical criteria for post-traumatic stress disorder. If so, this variable takes the value 1; if not, then 0.
Source: Part A and B claims

| Risk Factor | Mean | Minimum | Maximum |
|---|---|---|---|
| CCW indicator for post-traumatic | .006 | 0 | 1 |
| stress disorder | | | |

CCW indicator for pressure and chronic ulcers: For each person-month, this variable records whether the person meets the CCW clinical criteria for pressure and chronic ulcers. If so, this variable takes the value 1; if not, then 0.
Source: Part A and B claims

| Risk Factor | Mean | Minimum | Maximum |
|---|---|---|---|
| CCW indicator for pressure | .024 | 0 | 1 |
| and chronic ulcers | | | |

Indicator for problems with education and literacy: For each person-month, this variable records whether the person has incurred at least one inpatient or two non-inpatient claims with any diagnosis for problems with education and literacy within the past two years. If so, this variable takes the value 1; if not, then 0.
Source: Part A and B claims

| Risk Factor | Mean | Minimum | Maximum |
|---|---|---|---|
| Indicator for problems with | 0 | 0 | 1 |
| education and literacy | | | |

Indicator for problems with employment and unemployment: For each person-month, this variable records whether the person has incurred at least one inpatient or two non-inpatient claims with any diagnosis for problems with employment and unemployment within the past two years. If so, this variable takes the value 1; if not, then 0.
Source: Part A and B claims

| Risk Factor | Mean | Minimum | Maximum |
|---|---|---|---|
| Indicator for problems with employment | 0 | 0 | 1 |
| and unemployment | | | |

Indicator for problems with housing and economic conditions: For each person-month, this variable records whether the person has incurred at least one inpatient or two non-inpatient claims with any diagnosis for problems with housing and economic conditions within the past two years. If so, this variable takes the value 1; if not, then 0.
Source: Part A and B claims

| Risk Factor | Mean | Minimum | Maximum |
|---|---|---|---|
| Indicator for problems with housing | .001 | 0 | 1 |
| and economic conditions | | | |

Indicator for difficulty with life management: For each person-month, this variable records whether the person has incurred at least one inpatient or two non-inpatient claims with any diagnosis for difficulty with life management within the past two years. If so, this variable takes the value 1; if not, then 0.
Source: Part A and B claims

| Risk Factor | Mean | Minimum | Maximum |
|---|---|---|---|
| Indicator for difficulty with life | .002 | 0 | 1 |
| management | | | |

Indicator for lifestyle problems: For each person-month, this variable records whether the person has incurred at least one inpatient or two non-inpatient claims with any diagnosis for lifestyle problems within the past two years. If so, this variable takes the value 1; if not, then 0.
Source: Part A and B claims

| Risk Factor | Mean | Minimum | Maximum |
|---|---|---|---|
| Indicator for lifestyle problems | .021 | 0 | 1 |

Indicator for psychosocial problems: For each person-month, this variable records whether the person has incurred at least one inpatient or two non-inpatient claims with any diagnosis for psychosocial problems within the past two years. If so, this variable takes the value 1; if not, then 0.
Source: Part A and B claims

| Risk Factor | Mean | Minimum | Maxirnurn |
|---|---|---|---|
| Indicator for psychosocial | 0 | 0 | 1 |
| problems | | | |

Indicator for problems with social environment: For each person-month, this variable records whether the person has incurred at least one inpatient or two non-inpatient claims with any diagnosis for problems with social environment within the past two years. If so, this variable takes the value 1; if not, then 0.
Source: Part A and B claims

| Risk Factor | Mean | Minimum | Maximum |
|---|---|---|---|
| Indicator for problems | .002 | 0 | 1 |
| with social environment | | | |

Indicator for problems with upbringing: For each person-month, this variable records whether the person has incurred at least one inpatient or two non-inpatient claims with any diagnosis for problems with upbringing within the past two years. If so, this variable takes the value 1; if not, then 0.
Source: Part A and B claims

| Risk Factor | Mean | Minimum | Maximum |
|---|---|---|---|
| Indicator for problems | 0 | 0 | 1 |
| with upbringing | | | |

Indicator for problems with care provider dependency: For each person-month, this variable records whether the person has incurred at least one inpatient or two non-inpatient claims with any diagnosis for problems with care provider dependency within the past two years. If so, this variable takes the value 1; if not, then 0.
Source: Part A and B claims

| Risk Factor | Mean | Minimum | Maximum |
|---|---|---|---|
| Indicator for problems with | .047 | 0 | 1 |
| care provider dependency | | | |

CCW indicator for prostate cancer: For each person-month, this variable records whether the person meets the CCW clinical criteria for prostate cancer. If so, this variable takes the value 1; if not, then 0.
Source: Part A and B claims

| Risk Factor | Mean | Minimum | Maximum |
|---|---|---|---|
| CCW indicator for | .033 | 0 | 1 |
| prostate cancer | | | |

Indicator for protein-calorie malnutrition: For each person-month, this variable records whether the person has incurred at least one inpatient or two non-inpatient claims with any diagnosis for protein-calorie malnutrition within the past two years. If so, this variable takes the value 1; if not, then 0.
Source: Part A and B claims

| Risk Factor | Mean | Minimum | Maximum |
|---|---|---|---|
| Indicator for protein- | .005 | 0 | 1 |
| calorie malnutrition | | | |

Indicator for pulmonary circulatory disorder: For each person-month, this variable records whether the person has incurred at least one inpatient or two non-inpatient claims with any diagnosis for pulmonary circulatory disorder within the past two years. If so, this variable takes the value 1; if not, then 0.
Source: Part A and B claims

| Risk Factor | Mean | Minimum | Maximum |
|---|---|---|---|
| Indicator for pulmonary | .028 | 0 | 1 |
| circulatory disorder | | | |

CCW indicator for rheumatoid arthritis/osteoarthritis: For each person-month, this variable records whether the person meets the CCW clinical criteria for rheumatoid arthritis/osteoarthritis. If so, this variable takes the value 1; if not, then 0.
Source: Part A and B claims

| Risk Factor | Mean | Minimum | Maximum |
|---|---|---|---|
| CCW indicator for rheumatoid | .281 | 0 | 1 |
| arthritis/osteoarthritis | | | |

Indicator for respiratory infection: For each person-month, this variable records whether the person has incurred at least one inpatient or two non-inpatient claims with any diagnosis for respiratory infection within the past two years. If so, this variable takes the value 1; if not, then 0.
Source: Part A and B claims

| Risk Factor | Mean | Minimum | Maximum |
|---|---|---|---|
| Indicator for respiratory | .119 | 0 | 1 |
| infection | | | |

Indicator for retinopathy: For each person-month, this variable records whether the person has incurred at least one inpatient or two non-inpatient claims with any diagnosis for retinopathy within the past two years. If so, this variable takes the value 1; if not, then 0.
Source: Part A and B claims

| Risk Factor | Mean | Minimum | Maximum |
|---|---|---|---|
| Indicator for retinopathy | .001 | 0 | 1 |

Indicator for rheumatoid arthritis/collagen vascular disease: For each person-month, this variable records whether the person has incurred at least one inpatient or two non-inpatient claims with any diagnosis for rheumatoid arthritis/collagen vascular disease within the past two years. If so, this variable takes the value 1; if not, then 0.
Source: Part A and B claims

| Risk Factor | Mean | Minimum | Maximum |
|---|---|---|---|
| Indicator for rheumatoid | .048 | 0 | 1 |
| arthritis/collagen | | | |
| vascular disease | | | |

CCW indicator for schizophrenia and other psychotic disorders: For each person-month, this variable records whether the person meets the CCW clinical criteria for schizophrenia and other psychotic disorders. If so, this variable takes the value 1; if not, then 0.
Source: Part A and B claims

| Risk Factor | Mean | Minimum | Maximum |
|---|---|---|---|
| CCW indicator for schizophrenia | .012 | 0 | 1 |
| and other psychotic disorders | | | |

CCW indicator for sensory (blindness and visual) impairment: For each person-month, this variable records whether the person meets the CCW clinical criteria for sensory (blindness and visual) impairment. If so, this variable takes the value 1; if not, then 0.
Source: Part A and B claims

| Risk Factor | Mean | Minimum | Maximum |
|---|---|---|---|
| CCW indicator for sensory | .003 | 0 | 1 |
| (blindness and visual) | | | |
| impairment | | | |

CCW indicator for sensory (deafness and hearing) impairment: For each person-month, this variable records whether the person meets the CCW clinical criteria for sensory (deafness and hearing) impairment. If so, this variable takes the value 1; if not, then 0.
Source: Part A and B claims

| Risk Factor | Mean | Minimum | Maximum |
|---|---|---|---|
| CCW indicator for sensory | .046 | 0 | 1 |
| (deafness and hearing) | | | |
| impairment | | | |

Indicator for sepsis: For each person-month, this variable records whether the person has incurred at least one inpatient or two non-inpatient claims with any diagnosis for sepsis within the past two years. If so, this variable takes the value 1; if not, then 0.
Source: Part A and B claims

| Risk Factor | Mean | Minimum | Maximum |
|---|---|---|---|
| Indicator for sepsis | .018 | 0 | 1 |

Indicator for sleep apnea: For each person-month, this variable records whether the person has incurred at least one inpatient or two non-inpatient claims with any diagnosis for sleep apnea within the past two years. If so, this variable takes the value 1; if not, then 0.
Source: Part A and B claims

| Risk Factor | Mean | Minimum | Maximum |
|---|---|---|---|
| Indicator for sleep apnea | .108 | 0 | 1 |

Indicator for solid tumor without metastasis: For each person-month, this variable records whether the person has incurred at least one inpatient or two non-inpatient claims with any diagnosis for solid tumor without metastasis within the past two years. If so, this variable takes the value 1; if not, then 0.
Source: Part A and B claims

| Risk Factor | Mean | Minimum | Maximum |
|---|---|---|---|
| Indicator for solid tumor without metastasis | .094 | 0 | 1 |

CCW indicator for spina bifida and other congenital anomalies of the nervous system: For each person-month, this variable records whether the person meets the CCW clinical criteria for spina bifida and other congenital anomalies of the nervous system. If so, this variable takes the value 1; if not, then 0.
Source: Part A and B claims

| Risk Factor | Mean | Minimum | Maximum |
|---|---|---|---|
| CCW indicator for spina bifida and other | .001 | 0 | 1 |
| congenital anomalies of the nervous system | | | |

CCW indicator for spinal cord injury: For each person-month, this variable records whether the person meets the CCW clinical criteria for spinal cord injury. If so, this variable takes the value 1; if not, then 0.
Source: Part A and B claims

| Risk Factor | Mean | Minimum | Maximum |
|---|---|---|---|
| CCW indicator for spinal cord injury | .003 | 0 | 1 |

CCW indicator for stroke/ischemic transient attack: For each person-month, this variable records whether the person meets the CCW clinical criteria for stroke/ischemic transient attack. If so, this variable takes the value 1; if not, then 0.
Source: Part A and B claims

| Risk Factor | Mean | Minimum | Maximum |
|---|---|---|---|
| CCW indicator for stroke/ischemic | .032 | 0 | 1 |
| transient attack | | | |

CCW indicator for tobacco use: For each person-month, this variable records whether the person meets the CCW clinical criteria for tobacco use. If so, this variable takes the value 1; if not, then 0.
Source: Part A and B claims

| Risk Factor | Mean | Minimum | Maximum |
|---|---|---|---|
| CCW indicator for tobacco use | .054 | 0 | 1 |

CCW indicator for traumatic brain injury and nonpsychotic mental disorders due to brain damage: For each person-month, this variable records whether the person meets the CCW clinical criteria for traumatic brain injury and nonpsychotic mental disorders due to brain damage. If so, this variable takes the value 1; if not, then 0.
Source: Part A and B claims

| Risk Factor | Mean | Minimum | Maximum |
|---|---|---|---|
| CCW indicator for traumatic brain injury | .002 | 0 | 1 |
| and nonpsychotic mental disorders due to | | | |
| brain damage | | | |

Indicator for urinary tract infection: For each person-month, this variable records whether the person has incurred at least one inpatient or two non-inpatient claims with any diagnosis for urinary tract infection within the past two years. If so, this variable takes the value 1; if not, then 0.
Source: Part A and B claims

| Risk Factor | Mean | Minimum | Maximum |
|---|---|---|---|
| Indicator for urinary tract infection | .083 | 0 | 1 |

CCW indicator for viral hepatitis: For each person-month, this variable records whether the person meets the CCW clinical criteria for viral hepatitis. If so, this variable takes the value 1; if not, then 0.
Source: Part A and B claims

| Risk Factor | Mean | Minimum | Maximum |
|---|---|---|---|
| CCW indicator for viral hepatitis | .009 | 0 | 1 |

Indicator for cilostazol use: For each person-month, this variable takes the value of 1 if a person incurred a claim for cilostazol within the past 12 months, and 0 otherwise.
Source: Part D claims

| Risk Factor | Mean | Minimum | Maximum |
|---|---|---|---|
| Indicator for cilostazol use | .002 | 0 | 1 |

General internists per 1000 residents: For each person, this variable records the number of general internists per 1000 residents in the county containing the ZCTA. If the ZIP code tabulation area lies in two or more counties, the value is estimated as a weighted average of the county-level attributes, with weights being the fraction of the ZCTA population residing within each county.

### Source: Area Health Resources File

| Risk Factor | Mean | Minimum | Maximum |
|---|---|---|---|
| General internists per 1000 residents | .577 | 0 | 1.953442 |

Physician diversity: For each person, this variable records the percentage of medical doctors who are minorities (African Americans, Hispanics, and others, but excluding Asian-Americans). If the ZIP code tabulation area lies in two or more counties, the value is estimated as a weighted average of the county-level attributes, with weights being the fraction of the ZCTA population residing within each county.
Source: American Community Survey (2017, individual)

| Risk Factor | Mean | Minimum | Maximum |
|---|---|---|---|
| Physician diversity | 18.872 | 0 | 73.88705 |

Social workers per 1000 residents: For each person, this variable records the number of social workers per 1000 residents in the county containing the person's ZIP code tabulation area of residence. If the ZIP code tabulation area lies in two or more counties, the value is estimated as a weighted average of the county-level attributes, with weights being the fraction of the ZCTA population residing within each county.
Source: American Community Survey (2017, individual)

| Risk Factor | Mean | Minimum | Maximum |
|---|---|---|---|
| Social workers per 1000 residents | 3.772 | 0 | 6.382436 |

Indicator for frailty: For each person-month, this variable takes the value of 1 if a person meets the definition for frailty within the past twelve months, and 0 otherwise. The clinical definition for fraility is derived from Kim and Schneeweiss (2014).
Source: Part A, B, and Part B DME claims

| Risk Factor | Mean | Minimum | Maximum |
|---|---|---|---|
| Indicator for frailty | .268 | 0 | 1 |

Indicator for sickle cell anemia: For each person-month, this variable records whether the person has incurred at least one inpatient or two non-inpatient claims with any diagnosis for sickle cell anemia within the past two years. If so, this variable takes the value 1; if not, then 0.
Source: Part A and B claims

| Risk Factor | Mean | Minimum | Maximum |
|---|---|---|---|
| Indicator for sickle cell anemia | 0 | 0 | 1 |

Indicator for durable medical equipment (DME) use: For each person-month, this variable takes the value of 1 if a person used any durable medical equipment in the previous 12 months, and 0 otherwise.
Source: Part B DME claims

| Risk Factor | Mean | Minimum | Maximum |
|---|---|---|---|
| Indicator for durable medical | .257 | 0 | 1 |
| equipment (DME) use | | | |

Indicator for dual eligibility with Medicaid: For each person-month, this variable takes the value of 1 if a person was dually eligible for both Medicaid and Medicare within the past 12 months, and 0 otherwise.

### Source: Beneficiary Demographics

| Risk Factor | Mean | Minimum | Maximum |
|---|---|---|---|
| Indicator for dual | .134 | 0 | 1 |
| eligibility with Medicaid | | | |

Number of emergency department visits within the past 6 months: For each person-month, this variable counts the number of emergency department visits incurred within the prior 6 months.
Source: Part A claims

| Risk Factor | Mean | Minimum | Maximum |
|---|---|---|---|
| Number of emergency department | .176 | 0 | 100 |
| visits within the past 6 months | | | |

Indicator for endocrinologist visit: For each person-month, this variable takes the value of 1 if a person visited an endocrinologist within the past 12 months, and 0 otherwise.
Source: Part B claims

| Risk Factor | Mean | Minimum | Maximum |
|---|---|---|---|
| Indicator for endocrinologist visit | .07 | 0 | 1 |

Avoidable hospitalization/ED visit: For each person-month, this variable records whether the individual incurred an avoidable hospitalization or ED visits in that month. We use the AHRQ's definition of avoidable hospitalization in defining this outcome. Please see the section 3.2.1 of the documentation for additional detail.
Source: Part A claims

| Risk Factor | Mean | Minimum | Maximum |
|---|---|---|---|
| Avoidable hospitalization/ED visit | .006 | 0 | 1 |

Indicator for diabetic foot procedure: For each person-month, this variable takes the value of 1 if a person incurred an inpatient diabetic foot procedure over the last 12 months and 0 otherwise.
Source: Part A claims

| Risk Factor | Mean | Minimum | Maximum |
|---|---|---|---|
| Indicator for diabetic foot procedure | .001 | 0 | 1 |

Number of HbA1c tests: For each person-month, this variable counts the number of visits within the past 12 months in which a person received a Hemoglobin A1C (HbA1c) test. We define visits as unique combinations of person-provider-day.
Source: Part B claims

| Risk Factor | Mean | Minimum | Maximum |
|---|---|---|---|
| Number of HbA1c tests | .552 | 0 | 12 |

Number of heart-related procedures: For each person-month, this variable counts the number of heart-related procedures incurred over the past year.
Source: Part A claims

| Risk Factor | Mean | Minimum | Maximum |
|---|---|---|---|
| Number of heart-related procedures | .019 | 0 | 13 |

Number of home health visits: For each person-month, this variable counts the number of home health visits incurred within the past 12 months. We apply a logarithmic transformation to non-zero values. We define visits as unique combinations of person-provider-day.
Source: Part B claims

| Risk Factor | Mean | Minimum | Maximum |
|---|---|---|---|
| Number of home health visits | .015 | 0 | 5.56068 |

Indicator for hospice enrollment: For each person-month, this variable takes the value of 1 if a person enrolled in hospice within the past 12 months, and 0 otherwise.

### Source: Beneficiary Demographics

| Risk Factor | Mean | Minimum | Maximum. |
|---|---|---|---|
| Indicator for hospice enrollment | .001 | 0 | 1 |

Indicator for insulin use: For each person-month, this variable takes the value of 1 if a person incurred a claim for insulin within the past 12 months, and 0 otherwise.
Source: Part D claims

| Risk Factor | Mean | Minimum | Maximum |
|---|---|---|---|
| Indicator for insulin use | .04 | 0 | 1 |

Indicator for leukotriene receptor modifier use: For each person-month, this variable takes the value of 1 if a person incurred a claim for leukotriene receptor modifiers within the past 12 months, and 0 otherwise.
Source: Part D claims

| Risk Factor | Mean | Minimum | Maximum |
|---|---|---|---|
| Indicator for leukotriene | .03 | 0 | 1 |
| receptor modifier use | | | |

Indicator for no losartan use: For each person-month, this variable takes the value of 1 if a person did not incur a claim for losartan within the past 12 months, and 0 otherwise.
Source: Part D claims

| Risk Factor | Mean | Minimum | Maximum |
|---|---|---|---|
| Indicator for no losartan use | .892 | 0 | 1 |

Indicator for original Medicare eligibility for a non-age related cause: Beneficiary was originally eligible for Medicare for a reason other than age.

### Source: Beneficiary Demographics

| Risk Factor | Mean | Minimum | Maximum |
|---|---|---|---|
| Indicator for original Medicare | .166 | 0 | 1 |
| eligibility for a non-age related cause | | | |

Total health spending: For each person-month, this variable measures the total health spending incurred within the past 12 months. We define this as the sum of claim total charge amount (Part A), claim payment amount (Part B claim lines, aggregated to the claim level), and claim line beneficiary payment amount (part D).
Source: Part A, B, and D claims

| Risk Factor | Mean | Minimum | Maximum |
|---|---|---|---|
| Total health spending | 10712.08 | 0 | 3267903 |

Indicator for no mental health use: For each person-month, this variable takes the value of 1 if a person did not incur a visit with a mental health professional over the past 12 months, and 0 otherwise.
Source: Part B claims

| Risk Factor | Mean | Minimum | Maximum |
|---|---|---|---|
| Indicator for no | .948 | 0 | 1 |
| mental health use | | | |

Number of medications: For each person-month, this variable counts the number of distinct medications (as measured by NDC codes) for which there are part D claims within the past 12 months.
Source: Part D claims

| Risk Factor | Mean | Minimum | Maximum |
|---|---|---|---|
| Number of | 6.934 | 0 | 146 |
| medications | | | |

Indicator for oncologist visit: For each person-month, this variable takes the value of 1 if a person visited an oncologist within the past 12 months, and 0 otherwise.
Source: Part B claims

| Risk Factor | Mean | Minimum | Maximum |
|---|---|---|---|
| Indicator for | .095 | 0 | 1 |
| oncologist visit | | | |

Indicator for oral antibiotic use: For each person-month, this variable takes the value of 1 if a person incurred a claim for oral antibiotics within the past 12 months, and 0 otherwise.
Source: Part D claims

| Risk Factor | Mean | Minimum | Maximum |
|---|---|---|---|
| Indicator for oral | .301 | 0 | 1 |
| antibiotic use | | | |

Indicator for oral corticosteroid use: For each person-month, this variable takes the value of 1 if a person incurred a claim for oral corticosteroids within the past 12 months, and 0 otherwise.
Source: Part D claims

| Risk Factor | Mean | Minimum | Maximum |
|---|---|---|---|
| Indicator for oral | .055 | 0 | 1 |
| corticosteroid use | | | |

Number of outpatient visits: For each person-month, this variable counts the number of visits in an outpatient setting incurred within the past 12 months. We define visits as unique combinations of person-provider-day.
Source: Part B claims

| Risk Factor | Mean | Minimum | Maximum |
|---|---|---|---|
| Number of | 14.125 | 0 | 384 |
| outpatient visits | | | |

Continuity of primary care—Duration: For each person-month, this variable calculates the average time interval between primary care visits over the past 12 months. Visits that occur within 14 days are aggregated. Individuals with no primary care visits over the past 12 months are assigned a value of 365. We define visits as unique combinations of person-provider-day.
Source: Part B claims

| Risk Factor | Mean | Minimum | Maximum |
|---|---|---|---|
| Continuity of primary | 130.616 | 15.20833 | 365 |
| care-Duration | | | |

Discontinuity of primary care—Index: For each person-month, this variable calculates (1—the continuity of care index), from Boxerman and Bice (1977). This score ranges from 0 to 1 and is intended to measure dispersion in person-provider contact. If the person sees the same provider for all visits, indicating highly continuous care, the index score is 0; if the person sees a different physician for every visit, indicating fragmented care, the index score is 1. If a person has no primary care visits within the past year, they are assigned a value of 0. We define visits as unique combinations of person-provider-day.
Source: Part B claims

| Risk Factor | Mean | Minimum | Maximum |
|---|---|---|---|
| Discontinuity of | .62 | 0 | 1 |
| primary care-Index | | | |

Discontinuity of primary care—Proportion: For each person-month, this variable estimates (1—the fraction of primary care visits within the past 12 months provided by the same provider). For example, if a person had 10 primary care visits over the past 12 months, and four visits were with the same provider, then this measure would take a value of (1-0.4)=0.6. We define visits as unique combinations of person-provider-day.
Source: Part B claims

| Risk Factor | Mean | Minimum | Maximum |
|---|---|---|---|
| Discontinuity of primary | .545 | 0 | 1 |
| care-Proportion | | | |

Number of primary care visits: For each person-month, this variable counts the number of primary care visits within the past 12 months. We define visits as unique combinations of person-provider-day.
Source: Part B claims

| Risk Factor | Mean | Minimum | Maximum |
|---|---|---|---|
| Number of primary care visits | 8.799 | 0 | 315 |

Indicator for previous conservative diabetic wound procedure: For each person-month, this variable takes the value of 1 if a person underwent at least one conservative diabetic procedure within the past 12 months, and 0 otherwise.
Source: Part B claims

| Risk Factor | Mean | Minimum | Maximum |
|---|---|---|---|
| Indicator for previous conservative | .011 | 0 | 1 |
| diabetic wound procedure | | | |

Number of prior admissions: For each person-month, this variable counts the number of all inpatient hospital admissions incurred within the past twelve months.
Source: Part A claims

| Risk Factor | Mean | Minimum | Maximum |
|---|---|---|---|
| Number of prior admissions | .15 | 0 | 28 |

Prior admission length of stay: For each person-month, this variable calculates the length of the most recently incurred hospital inpatient stay over the past 12 months. For individuals without a previous inpatient stay, this value is set to zero.
Source: Part A claims

| Risk Factor | Mean | Minimum | Maximum |
|---|---|---|---|
| Prior admission length of stay | .456 | 0 | 436 |

Prior hospitalization admission source—none: For each person-month, this variable indicates the individual did not incur an inpatient hospital stay within the past 12 month.

### Source: Part A Claims

| Risk Factor | Mean | Minimum | Maximum |
|---|---|---|---|
| Prior hospitalization | .898 | 0 | 1 |
| admission source-none | | | |

Prior hospitalization admission source—other: For each person-month, this variable indicates that for the individual's most recently incurred inpatient hospital stay within the past 12 months, the individual's admission source was: other.

### Source: Part A Claims

| Risk Factor | Mean | Minimum | Maximum |
|---|---|---|---|
| Prior hospitalization | 0 | 0 | 0 |
| admission source-other | | | |

Prior hospitalization admission source—physician referral: For each person-month, this variable indicates that for the individual's most recently incurred inpatient hospital stay within the past 12 months, the individual's admission source was: physician referral.

### Source: Part A Claims

| Risk Factor | Mean | Minimum | Maximum |
|---|---|---|---|
| Prior hospitalization admission | .091 | 0 | 1 |
| source-physician referral | | | |

Prior hospitalization admission source—transferred from facility: For each person-month, this variable indicates that for the individual's most recently incurred inpatient hospital stay within the past 12 months, the individual's admission source was: transferred from facility.

### Source: Part A Claims

| Risk Factor | Mean | Minimum | Maximum |
|---|---|---|---|
| Prior hospitalization admission | .011 | 0 | 1 |
| source-transferred from facility | | | |

Prior hospitalization admission type—elective: For each person-month, this variable indicates that for the individual's most recently incurred inpatient hospital stay within the past 12 months, the individual's admission type was: elective.

### Source: Part A Claims

| Risk Factor | Mean | Minimum | Maximum |
|---|---|---|---|
| Prior hospitalization admission | .031 | 0 | 1 |
| type-elective | | | |

Prior hospitalization admission type—emergency: For each person-month, this variable indicates that for the individual's most recently incurred inpatient hospital stay within the past 12 months, the individual's admission type was: emergency.

### Source: Part A Claims

| Risk Factor | Mean | Minimum | Maximum |
|---|---|---|---|
| Prior hospitalization admission | .066 | 0 | 1 |
| type-emergency | | | |

Prior hospitalization admission type—none: For each person-month, this variable indicates the individual did not incur an inpatient hospital stay within the past 12 month.

### Source: Part A Claims

| Risk Factor | Mean | Minimum | Maximum |
|---|---|---|---|
| Prior hospitalization admission | .898 | 0 | 1 |
| type-none | | | |

Prior hospitalization admission type—other: For each person-month, this variable indicates that for the individual's most recently incurred inpatient hospital stay within the past 12 months, the individual's admission type was: other.

### Source: Part A Claims

| Risk Factor | Mean | Minimum | Maximum |
|---|---|---|---|
| Prior hospitalization admission type-other | 0 | 0 | 1 |

Prior hospitalization admission type—trauma center: For each person-month, this variable indicates that for the individual's most recently incurred inpatient hospital stay within the past 12 months, the individual's admission type was: trauma center.

### Source: Part A Claims

| Risk Factor | Mean | Minimum | Maximum |
|---|---|---|---|
| Prior hospitalization admission | .001 | 0 | 1 |
| type-trauma center | | | |

Prior hospitalization admission type—urgent: For each person-month, this variable indicates that for the individual's most recently incurred inpatient hospital stay within the past 12 months, the individual's admission type was: urgent.

### Source: Part A Claims

| Risk Factor | Mean | Minimum | Maximum |
|---|---|---|---|
| Prior hospitalization admission | .004 | 0 | 1 |
| type-urgent | | | |

Prior hospitalization discharge status—home: For each person-month, this variable indicates that for the individual's most recently incurred inpatient hospital stay within the past 12 months, the individual's discharge status was: home.

### Source: Part A Claims

| Risk Factor | Mean | Minimum | Maximum |
|---|---|---|---|
| Prior hospitalization discharge | .082 | 0 | 1 |
| status-home | | | |

Prior hospitalization discharge status—none: For each person-month, this variable indicates the individual did not incur an inpatient hospital stay within the past 12 month.

### Source: Part A Claims

| Risk Factor | Mean | Minimum | Maximum |
|---|---|---|---|
| Prior hospitalization discharge | .898 | 0 | 1 |
| status-none | | | |

Prior hospitalization discharge status—other: For each person-month, this variable indicates that for the individual's most recently incurred inpatient hospital stay within the past 12 months, the individual's discharge status was: other.

### Source: Part A Claims

| Risk Factor | Mean | Minimum | Maximum |
|---|---|---|---|
| Prior hospitalization discharge status-other | 0 | 0 | 1 |

Prior hospitalization discharge status—transferred to inpatient care: For each person-month, this variable indicates that for the individual's most recently incurred inpatient hospital stay within the past 12 months, the individual's discharge status was: transferred to inpatient care.

### Source: Part A Claims

| Risk Factor | Mean | Minimum | Maximum |
|---|---|---|---|
| Prior hospitalization discharge | 0 | 0 | 0 |
| status-transferred to inpatient care | | | |

Prior hospitalization discharge status—transferred to post-acute care: For each person-month, this variable indicates that for the individual's most recently incurred inpatient hospital stay within the past 12 months, the individual's discharge status was: transferred to post-acute care.

### Source: Part A Claims

| Risk Factor | Mean | Minimum | Maximum |
|---|---|---|---|
| Prior hospitalization discharge | 0 | 0 | 0 |
| status-transferred to post-acute care | | | |

Indicator for prior nursing home stay: For each person-month, this variable takes the value of 1 if a person incurred a nursing home stay within the last 12 months, and 0 otherwise.
Source: Part A claims

| Risk Factor | Mean | Minimum | Maximum |
|---|---|---|---|
| Indicator for prior nursing home stay | .024 | 0 | 1 |

Indicator for prior readmission: For each person-month, this variable takes the value of 1 if a person incurred an all-cause 30-day hospital readmission within the last 12 months, and 0 otherwise. We define readmission as two inpatient stays occurring fewer than 30 days apart.
Source: Part A claims

| Risk Factor | Mean | Minimum | Maximum |
|---|---|---|---|
| Indicator for prior readmission | .018 | 0 | 1 |

Indicator for prior surgery: For each person-month, this variable takes the value of 1 if a person underwent a surgery within the past 12 months, and 0 otherwise.
Source: Part B claims

| Risk Factor | Mean | Minimum | Maximum |
|---|---|---|---|
| Indicator for prior surgery | .563 | 0 | 1 |

Indicator for provider administered drug: For each person-month, this variable takes the value of 1 if a person received a provider-administered drug as defined by a ‘J code’ in the past 12 months, and 0 otherwise.
Source: Part B claims

| Risk Factor | Mean | Minimum | Maximum |
|---|---|---|---|
| Indicator for provider | .214 | 0 | 1 |
| administered drug | | | |

Beneficiary race—Asian: Beneficiary's Research Triangle Institute (RTI) race code is Asian.

### Source: Beneficiary Demographics

| Risk Factor | Mean | Minimum | Maximum |
|---|---|---|---|
| Beneficiary race-Asian | .019 | 0 | 1 |

Beneficiary race—Black: Beneficiary's Research Triangle Institute (RTI) race code is Black.

### Source: Beneficiary Demographics

| Risk Factor | Mean | Minimum | Maximum |
|---|---|---|---|
| Beneficiary race-Black | .215 | 0 | 1 |

Beneficiary race—Hispanic: Beneficiary's Research Triangle Institute (RTI) race code is Hispanic.

### Source: Beneficiary Demographics

| Risk Factor | Mean | Minimum | Maximum |
|---|---|---|---|
| Beneficiary race-Hispanic | .009 | 0 | 1 |

Beneficiary race—Native American: Beneficiary's Research Triangle Institute (RTI) race code is Native American.

### Source: Beneficiary Demographics

| Risk Factor | Mean | Minimum | Maximum |
|---|---|---|---|
| Beneficiary race- | .001 | 0 | 1 |
| Native American | | | |

Beneficiary race—Other: Beneficiary's Research Triangle Institute (RTI) race code is Other.

### Source: Beneficiary Demographics

| Risk Factor | Mean | Minimum | Maximum |
|---|---|---|---|
| Beneficiary race-Other | .016 | 0 | 1 |

Beneficiary race—Unknown: Beneficiary's Research Triangle Institute (RTI) race code is Unknown.

### Source: Beneficiary Demographics

| Risk Factor | Mean | Minimum | Maximum |
|---|---|---|---|
| Beneficiary race-Unknown | 024 | 0 | 1 |

Beneficiary race—White: Beneficiary's Research Triangle Institute (RTI) race code is White.

### Source: Beneficiary Demographics

| Risk Factor | Mean | Minimum | Maximum |
|---|---|---|---|
| Beneficiary race-White | 717 | 0 | 1 |

Indicator for rivaroxaban use: For each person-month, this variable takes the value of 1 if a person incurred a claim for rivaroxaban within the past 12 months, and 0 otherwise.
Source: Part D claims

| Risk Factor | Mean | Minimum | Maximum |
|---|---|---|---|
| Indicator for | .017 | 0 | 1 |
| rivaroxaban use | | | |

Number of rural clinic visits: For each person-month, this variable counts the number of rural clinic visits incurred within the past 12 months. We define visits as unique combinations of person-provider-day.
Source: Part B claims

| Risk Factor | Mean | Minimum | Maximum |
|---|---|---|---|
| Number of rural clinic visits | 0 | 0 | 1 |

Beneficiary gender—female: Beneficiary gender is female.

### Source: Beneficiary Demographics

| Risk Factor | Mean. | Minimum | Maximum |
|---|---|---|---|
| Beneficiary gender-female | .596 | 0 | 1 |

Beneficiary gender—male: Beneficiary gender is male.

### Source: Beneficiary Demographics

| Risk Factor | Mean | Minimum | Maximum |
|---|---|---|---|
| Beneficiary gender-male | .404 | 0 | 1 |

Number of specialist visits: For each person-month, this variable counts the number of specialist visits incurred within the past 12 months. We define visits as unique combinations of person-provider-day.
Source: Part B claims

| Risk Factor | Mean | Minimum | Maximum |
|---|---|---|---|
| Number of specialist visits | 4.889 | 0 | 365 |

Indicator for no statin use: For each person-month, this variable takes the value of 1 if a person did not incur a claim for statins within the past 12 months, and 0 otherwise. Source: Part D claims

| Risk Factor | Mean | Minimum | Maximum |
|---|---|---|---|
| Indicator for no statin use | .644 | 0 | 1 |

Number of lab tests: For each person-month, this variable counts the number of visits within the past 12 months in which a person received any laboratory test. We define visits as unique combinations of person-provider-day.
Source: Part B claims

| Risk Factor | Mean | Minimum | Maximum |
|---|---|---|---|
| Number of lab tests | .146 | 0 | 53 |

Number of urgent care visits: For each person-month, this variable counts the number of urgent care visits incurred within the past 12 months. We define visits as unique combinations of person-provider-day.
Source: Part B claims

| Risk Factor | Mean | Minimum | Maximum |
|---|---|---|---|
| Number of urgent | .148 | 0 | 79 |
| care visits | | | |

Indicator for no vaccination (flu or pneumonia): For each person-month, this variable takes the value of 1 if a person did not receive a vaccination (flu or pneumonia) within the past 12 months, 0 otherwise.
Source: Part B claims

| Risk Factor | Mean | Minimum | Maximum |
|---|---|---|---|
| Indicator for no vaccination | .482 | 0 | 1 |
| (flu or pneumonia) | | | |

Indicator for warfarin use: For each person-month, this variable takes the value of 1 if a person incurred a claim for warfarin within the past 12 months, and 0 otherwise.
Source: Part D claims

| Risk Factor | Mean | Minimum | Maximum |
|---|---|---|---|
| Indicator for warfarin use | .024 | 0 | 1 |

Number of hospital beds per 1000 residents: For each person, this variable records the number of active (short term or critical access or transplant) hospital beds per 1000 residents in the person's ZIP code tabulation area of residence.
Source: CMS Provider of Service Files (December 2018) American Community Survey (2017, 5 year estimates)

| Risk Factor | Mean | Minimum | Maximum |
|---|---|---|---|
| Number of hospital | 1.782 | 0 | 69.72138 |
| beds per 1000 residents | | | |

National ranking of deprivation: For each person, this variable records the national ranking of deprivation for the person's ZIP code tabulation area of residence. This index ‘includes factors for the theoretical domains of income, education, employment, and housing quality.’ See https://www.neighborhoodatlas.medicine.wisc.edu/ for additional detail. Higher values indicate a greater degree of deprivation.

### Source: Neighborhood Atlas

| Risk Factor | Mean | Minimum | Maximum |
|---|---|---|---|
| National ranking of deprivation | 28.595 | 0 | 94.28051 |

Indicator for presence of a for-profit hospital: For each person, this variable records whether the person's ZIP code tabulation area of residence contains at least one active (short term or critical access or transplant) for-profit hospital.

### Source: CMS Provider of Service Files (December 2018)

| Risk Factor | Mean | Minimum | Maximum |
|---|---|---|---|
| Indicator for presence of a | .004 | 0 | 1 |
| for-profit hospital | | | |

Indicator for no federally qualified health center: For each person, this variable records whether the person's ZIP code tabulation area of residence does not contain at least one active federally qualified health center.

### Source: CMS Provider of Service Files (December 2018)

| Risk Factor | Mean | Minimum | Maximum |
|---|---|---|---|
| Indicator for no federally | .752 | 0 | 1 |
| qualified health center | | | |

Indicator for no mental health center: For each person, this variable records whether the person's ZIP code tabulation area of residence does not contain at least one active community mental health center.

### Source: CMS Provider of Service Files (December 2018)

| Risk Factor | Mean | Minimum | Maximum |
|---|---|---|---|
| Indicator for no | .978 | 0 | 1 |
| mental health center | | | |

Indicator for no rural health clinic: For each person, this variable records whether the person's ZIP code tabulation area of residence does not contain at least one active rural health clinic.

### Source: CMS Provider of Service Files (December 2018)

| Risk Factor | Mean | Minimum | Maximum |
|---|---|---|---|
| Indicator for no | 1 | 0 | 1 |
| rural health clinic | | | |

Indicator for no VA clinic or VA medical center: For each person, this variable records whether the person's ZIP code tabulation area of residence does not contain at least one VA clinic or medical center.

### Source: Veterans Affairs Facility Listing

| Risk Factor | Mean | Minimum | Maximum |
|---|---|---|---|
| Indicator for no VA clinic | .936 | 0 | 1 |
| or VA medical center | | | |

Number of hospitals per 1000 residents: For each person, this variable records the number of active (short term or critical access or transplant) hospitals per 1000 residents in the person's ZIP code tabulation area of residence. Source: CMS Provider of Service Files (December 2018) American Community Survey (2017, 5 year estimates)

| Risk Factor | Mean | Minimum | Maximum |
|---|---|---|---|
| Number of hospitals | .009 | 0 | .3377237 |
| per 1000 residents | | | |

Median household income: For each person, this variable records the median household income in the person's ZIP code tabulation area of residence (pooled from 2013-2017).
Source: American Community Survey (2017. 5 year estimates)

| Risk Factor | Mean | Minimum | Maximum |
|---|---|---|---|
| Median household income | 84094.19 | 16188 | 218638 |

Located in partial county mental health care shortage area: For each person, this variable takes the value of 1 if the person's ZIP code tabulation area of residence is located in a county that is designated by HRSA in 2018 to be a partial-county mental health care shortage area. The variable takes the value of 0, otherwise. If the ZIP code tabulation area lies in two counties, the value is estimated as a weighted average of the county-level attributes.

### Source: Area Health Resources File

| Risk Factor | Mean | Minimum | Maximum |
|---|---|---|---|
| Located in partial county | .65 | 0 | 1.0001 |
| mental health care shortage area | | | |

Located in whole county mental health care shortage area: For each person, this variable takes the value of 1 if the person's ZIP code tabulation area of residence is located in a county that is designated by HRSA in 2018 to be a whole-county mental health care shortage area. The variable takes the value of 0, otherwise. If the ZIP code tabulation area lies in two or more counties, the value is estimated as a weighted average of the county-level attributes.

### Source: Area Health Resources File

| Risk Factor | Mean | Minimum | Maximum |
|---|---|---|---|
| Located in whole county | .218 | 0 | 1.0001 |
| mental health care shortage area | | | |

Number of hospitals: For each person, this variable records the number of active (short term or critical access or transplant) hospitals in the person's ZIP code tabulation area of residence.

### Source: CMS Provider of Service Files (December 2018)

| Risk Factor | Mean | Minimum | Maximum |
|---|---|---|---|
| Number of hospitals | .259 | 0 | 4 |

Number of primary care physicians per 1000 residents: For each person, this variable records the number of primary care physicians per 1000 residents in the person's ZIP code tabulation area of residence.
Source: AMA, American Community Survey (2017, 5 year estimates)

| Risk Factor | Mean | Minimum | Maximum |
|---|---|---|---|
| Number of primary care physicians | .857 | 0 | 11.47018 |
| per 1000 residents | | | |

Percent aged 65 and over: For each person, this variable records the percentage of individuals in the person's ZIP code tabulation area of residence aged 65 and over (pooled from 2013-2017).
Source: American Community Survey (2017, 5 year estimates)

| Risk Factor | Mean | Minimum | Maximum |
|---|---|---|---|
| Percent aged 65 and over | 15.774 | 0 | 100 |

Percent with less than high school education, ages 65+: For each person, this variable records the percent of the population aged 65 and above in the person's ZIP code tabulation area that has less than a high school diploma.
Source: American Community Survey (2017, 5 year estimates)

| Risk Factor | Mean | Minimum | Maximum |
|---|---|---|---|
| Percent with less than high school | 15.032 | 0 | 100 |
| education, ages 65+ | | | |

Percent live alone, ages 65+: For each person, this variable records the percent of the population aged 65 and above in the person's ZIP code tabulation area that lives alone.
Source: American Community Survey (2017, 5 year estimates)

| Risk Factor | Mean | Minimum | Maximum |
|---|---|---|---|
| Percent live alone, ages 65+ | 26.364 | 0 | 100 |

Percent speak Spanish, aged 65+: For each person, this variable records the percent of the population aged 65 and above in the person's ZIP code tabulation area that speaks Spanish.
Source: American Community Survey (2017. 5 year estimates)

| Risk Factor | Mean | Minimum | Maximum |
|---|---|---|---|
| Percent speak Spanish, aged 65+ | 2.363 | 0 | 92.90981 |

Percent in poverty age 65+: For each person, this variable records the percentage of people age 65+whose income in the past 12 months is below the poverty level in the person's ZIP code tabulation area of residence (pooled from 2013-2017).
Source: American Community Survey (2017, 5 year estimates)

| Risk Factor | Mean | Minimum | Maximum |
|---|---|---|---|
| Percent in poverty age 65+ | 7.646 | 0 | 100 |

Percent foreign born: For each person, this variable records the percent of individuals who are foreign-born in the person's ZIP code tabulation area of residence.
Source: American Community Survey (2017, 5 year estimates)

| Risk Factor | Mean | Minimum | Maximum |
|---|---|---|---|
| Percent foreign born | 12.233 | 0 | 66.4828 |

Percent Hispanic, ages 65+: For each person, this variable records the percent of the population aged 65 and above in the person's ZIP code tabulation area that is Hispanic.
Source: American Community Survey (2017, 5 year estimates)

| Risk Factor | Mean | Minimum | Maxirnurn |
|---|---|---|---|
| Percent Hispanic, ages 65+ | 2.551 | 0 | 100 |

Percent with less than high school education: For each person, this variable records the percent of individuals age 18 and older with less than a high school diploma in the person's ZIP code tabulation area of residence.
Source: American Community Survey (2017, 5 year estimates)

| Risk Factor | Mean | Minimum | Maximum. |
|---|---|---|---|
| Percent with less than high | 9.564 | 0 | 48 |
| school education | | | |

Percent married: For each person, this variable records the percent of the population aged 15+ in the person's ZIP code tabulation area of residence that is currently married (pooled from 2013-2017).
Source: American Community Survey (2017, 5 year estimates)

| Risk Factor | Mean | Minimum | Maximum |
|---|---|---|---|
| Percent married | 49.078 | 0 | 100 |

Percent Native American: For each person, this variable records the percent of the population in the person's ZIP code tabulation area that is Native American.
Source: American Community Survey (2017, 5 year estimates)

| Risk Factor | Mean | Minimum | Maximum |
|---|---|---|---|
| Percent Native American | .242 | 0 | 17.21282 |

Percent non-English speakers: For each person, this variable records the percent of individuals who speak Spanish or other languages and who speak English less than ‘very well’ in the person's ZIP code tabulation area of residence.
Source: American Community Survey (2017, 5 year estimates)

| Risk Factor | Mean | Minimum | Maximum |
|---|---|---|---|
| Percent non-English speakers | 5.284 | 0 | 48.36707 |

Percent non-white, ages 65+: For each person, this variable records the percent of the population aged 65 and above in the person's ZIP code tabulation area that is non-white.
Source: American Community Survey (2017, 5 year estimates)

| Risk Factor | Mean | Minimum | Maximum |
|---|---|---|---|
| Percent non-white, ages 65+ | 26.864 | 0 | 100 |

Percent in poverty: For each person, this variable records the percentage of families whose income in the past 12 months is below the poverty level in the person's ZIP code tabulation area of residence (pooled from 2013-2017) .
Source: American Community Survey (2017, 5 year estimates)

| Risk Factor | Mean | Minimum | Maximum |
|---|---|---|---|
| Percent in poverty | 6.563 | 0 | 50 |

Percent single mothers: For each person, this variable records the percent of women aged 15-50 giving birth within the past 12 months who are not married in the person's ZIP code tabulation area
Source: American Community Survey (2017, 5 year estimates)

| Risk Factor | Mean | Minimum | Maximum |
|---|---|---|---|
| Percent single mothers | 32.162 | 0 | 100 |

Percent aged 0-4: For each person, this variable records the percentage of individuals in the person's ZIP code tabulation area of residence aged 0-4 (pooled from 2013-2017).
Source: American Community Survey (2017, 5 year estimates)

| Risk Factor | Mean | Minimum | Maximum |
|---|---|---|---|
| Percent aged 0-4 | 5.803 | 0 | 33.15217 |

Number of pharmacies per 1000 population: For each person, this variable records the number of active pharmacies per 1000 population in the person's ZIP code tabulation area of residence.

### Source: Maryland Board of Pharmacies

| Risk Factor | Mean | Minimum | Maximum |
|---|---|---|---|
| Number of pharmacies | .23 | 0 | 1.205182 |
| per 1000 population | | | |

Air pollution level: For each person, this variable records the average daily fine particulate matter (PM 2.5) concentration from the EPA's Downscaler Model for 2011-2014 in the person's ZIP code tabulation area of residence.

### Source: Environmental Protection Agency

| Risk Factor | Mean | Minimum | Maximum |
|---|---|---|---|
| Air pollution level | 9.689 | 0 | 12.41805 |

Population: For each person, this variable records the population of the person's ZIP code tabulation area of residence.
Source: American Community Survey (2017, 5 year estimates)

| Risk Factor | Mean | Minimum | Maximum |
|---|---|---|---|
| Population | 30658.23 | 0 | 119204 |

Population growth: For each person, this variable records the percent population growth recorded in the person's ZIP code tabulation area of residence from 2011-2017.
Source: American Community Survey (2011 and 2017, 5 year estimates)

| Risk Factor | Mean | Minimum | Maximum |
|---|---|---|---|
| Population growth | 4.705 | −100 | 475 |

Population density: For each person, this variable records the population per square mile in the person's ZIP code tabulation area of residence.
Source: American Community Survey (2017, 5 year estimates), Census

| Risk Factor | Mean | Minimum | Maximum |
|---|---|---|---|
| Population density | 2614.176 | 0 | 97473.26 |

Located in partial county primary care shortage area: For each person, this variable takes the value of 1 if the person's ZIP code tabulation area of residence is located in a county that is designated by HRSA in 2018 to be a partial-county primary care shortage area. The variable takes the value of 0, otherwise. If the ZIP code tabulation area lies in two or more counties, the value is estimated as a weighted average of the county-level attributes.

### Source: Area Health Resources File

| Risk Factor | Mean | Minimum | Maximum |
|---|---|---|---|
| Located in partial county | .888 | 0 | 1.0001 |
| primary care shortage area | | | |

Located in whole county primary care shortage area: For each person, this variable takes the value of 1 if the person's ZIP code tabulation area of residence is located in a county that is designated by HRSA in 2018 to be a whole-county primary care shortage area. The variable takes the value of 0, otherwise. If the ZIP code tabulation area lies in two or more counties, the value is estimated as a weighted average of the county-level attributes.

### Source: Area Health Resources File

| Risk Factor | Mean | Minimum | Maximum |
|---|---|---|---|
| Located in whole county | 0 | 0 | 1 |
| primary care shortage area | | | |

Rurality index: For each person, this variable records the rural/urban index for the person's ZIP code tabulation area of residence. This data is comprised of 10 codes which “delineate metropolitan, micropolitan, small town, and rural commuting areas based on the size and direction of the primary (largest) commuting flows.” Higher values indicate a greater degree of rurality.

### Source: USDA Rural-Urban Commuting Area Codes

| Risk Factor | Mean | Minimum | Maximum |
|---|---|---|---|
| Rurality index | 1.438 | 0 | 10 |

Number of specialty care physicians per 1000 residents: For each person, this variable records the number of specialty care physicians per 1000 residents in the person's ZIP code tabulation area of residence.
Source: AMA, American Community Survey (2017, 5 year estimates)

| Risk Factor | Mean | Minimum | Maximum |
|---|---|---|---|
| Number of specialty care | 1.682 | 0 | 44.88318 |
| physicians per 1000 residents | | | |

Taxable interest per capita: For each person, this variable records taxable interest (tax year 2016) per person in the person's ZIP code tabulation area of residence.
Source: IRS Statistics of Income and American Community Survey (2017, 5 year estimates)

| Risk Factor | Mean | Minimum | Maximum |
|---|---|---|---|
| Taxable interest per capita | 225.267 | 0 | 5886.03 |

Walkability index: For each person, this variable records the value of the National Walkability Index for the person's ZIP code tabulation area of residence.

### Source: Environmental Protection Agency

| Risk Factor | Mean | Minimum | Maximum |
|---|---|---|---|
| Walkability index | 9.801 | 0 | 18.98414 |

### APPENDIX 1: HEDIS ANTIBIOTICS TABLES

### Antibiotics of Concern by NCQA Drug Class Medications

| Description | Prescriptions | | |
|---|---|---|---|
| Quinolone | Ciprofloxacin | Levofloxacin | Norfloxacin |
| | Gemifloxacin | Moxifloxacin | Ofloxacin |
| Azithromycin and | Azithromycin | Clarithromycin | |
| clarithromycin | | | |
| Cephalosporin | Cefaclor | Cefotaxime | Ceftriaxone |
| (second, third, | Cefdinir | Cefotetan | Cefuroxime |
| fourth generation) | Cefditoren | Cefoxitin | Ceftazidime |
| | Cefepime | Cefpodoxime | Ceftibuten |
| | Cefixime | Cefprozil | |
| Amoxicillin/ | Amoxicillin- | | |
| clavulanate | clavulanate | | |
| Ketolide | Telithromycin | | |
| Clindamycin | Clindamycin | | |
| Miscellaneous | Aztreonam | Dalfopristin- | Telavancin |
| antibiotics of | Chloramphenicol | quinupristin | Vancomycin |
| concern | | Linezolid | |

### All Other Antibiotics by NCQA Drug Class Medications

| Description | Prescriptions | |
|---|---|---|
| Absorbable | Sulfadiazine | Sulfamethoxazole- |
| sulfonamide | | trimethoprim |
| Aminoglycoside | Amikacin | Streptomycin |
| | Gentamicin | Tobramycin |
| Cephalosporin | Cefadroxil | Cephalexin |
| (first generation) | Cefazolin | |
| Lincosamide | Lincomycin | |
| (other than | | |
| clindamycin) | | |
| Macrolide | Erythromycin | Erythromycin stearate |
| (other than | Erythromycin | Erythromycin-sulfisoxazole |
| azithromycin and | ethylsuccinate | |
| clarithromycin) | Erythromycin | |
| | lactobionate | |
| Penicillin | Ampicillin | Penicillin G potassium |
| (other than | Ampicillin-sulbactam | Penicillin G procaine |
| amoxicillin/ | Amoxicillin | Penicillin G sodium |
| clavulanate) | Dicloxacillin | Penicillin V potassium |
| | Nafcillin | Piperacillin-tazobactam |
| | Oxacillin | Ticarcillin-clavulanate |
| | Penicillin G | |
| | benzathine | |
| Tetracyclines | Doxycyline | Tetracycline |
| | Minocycline | |
| Miscellaneous | Daptomycin | Nitrofurantoin macrocrystals |
| antibiotics | Fosfomycin | Rifampin |
| | Metronidazole | Trimethoprim |
| | Nitrofurantoin | |

### REFERENCES

Alba, A. C., Agoritsas, T., Walsh, M., Hanna, S., Iorio, A., Devereaux, P. J., McGinn, T., & Guyatt, G. (2017.) Discrimination and calibration of clinical prediction models: Users' guides to the medical literature. JAMA 318(14), 1377-1384.
Andersen, R. M. (1995). Revisiting the behavioral model and access to medical care: Does it matter? Journal of health and social behavior, 1-10.
Anderson, G. F., Ballreich, J., Bleich, S., Boyd, C., DuGoff, E., Leff, B., Salzburg, C., & Wolff, J. (2015). Attributes common to programs that successfully treat high-need, high-cost individuals. The American Journal of Managed Care, 21(11), e597-600.
Bagherzadeh-Khiabani, F., Ramezankhani, A., Azizi, F., Hadaegh, F., Steyerberg, E. W., & Khalili, D. (2016). A tutorial on variable selection for clinical prediction models: Feature selection methods in data mining could improve the results. Journal of Clinical Epidemiology, 71, 76-85.
Baker, J. M., Grant, R. W., & Gopalan, A. (2018). A systematic review of care management interventions targeting multimorbidity and high care utilization. BMC Health Services Research, 18(1), 65.
Berkowitz, S. A., Parashuram, S., Rowan, K., Andon, L., Bass, E. B., Bellantoni, M., Brotman, D. J., et al. (2018). Association of a care coordination model with health care costs and utilization: The Johns Hopkins Community Health Partnership (J-CHiP). JAMA Network Open, 1(7), e184273-e184273.
Billings, J., Zeitel, L., Lukomnik, J., Carey, T. S., Blank, A. E., & Newman, L. (1993). Impact of socioeconomic status on hospital use in New York City. Health Affairs, 12(1), 162-173.
Brophy, J. M., Joseph, L., & Rouleau, J. L. (2001). β-Blockers in congestive heart failure: A Bayesian meta-analysis. Annals of Internal Medicine, 134(7), 550-560.
Center for Medicare & Medicaid Innovation. (2017, December). CPC+ payment methodologies: Beneficiary attribution, care management fee, performance-based incentive payment, and payment under the Medicare physician fee schedule. Version 1. Retrieved from https://innovation.cms.gov/Files/x/cpcplus-methodology.pdf
Center for Medicare & Medicaid Innovation. (2019, April). MDPCP payment methodologies: Beneficiary attribution, care management fee, performance-based incentive payment, and comprehensive care payment. Version 1.0p.
Centers for Medicare & Medicaid Services. (2018, December). Report to Congress: Risk adjustment in Medicare Advantage. Retrieved from https://www.cms.gov/Medicare/Health-Plans/MedicareAdvtgSpecRateStats/Downloads/RTC-Dec2018.pdf
Congressional Research Service. (2006). Changing postal ZIP code boundaries. Retrieved from https://apps.dtic.mil/dtic/tr/fulltext/u²/ₐ462043.pdf
Edwards, S. T., Peterson, K., Chan, B., Anderson, J., & Helfand, M. (2017). Effectiveness of intensive primary care interventions: A systematic review. Journal of General Internal Medicine, 32(12), 1377-1386.
Furumoto, A., Ohkusa, Y., Chen, M., Kawakami, K., Masaki, H., Sueyasu, Y., . . . & Oishi, K. (2008). Additive effect of pneumococcal vaccine and influenza vaccine on acute exacerbation in patients with chronic lung disease. Vaccine, 26(33), 4284-4289.
Hammill, B. G., Curtis, L. H., Fonarow, G. C., Heidenreich, P. A., Yancy, C. W., Peterson, E. D., & Hernandez, A. F. (2011). Incremental value of clinical data beyond claims data in predicting 30-day outcomes after heart failure hospitalization. Circulation: Cardiovascular Quality and Outcomes, 4(1), 60-67.
Hedlund, J., Christenson, B., Lundbergh, P., & Örtqvist, Å. (2003). Effects of a large-scale intervention with influenza and 23-valent pneumococcal vaccines in elderly people: A 1-year follow-up. Vaccine, 21(25-26), 3906-3911.
Hong, C. S., Siegel, A. L., & Ferris, T. G. (2014). Caring for high-need, high-cost patients: What makes for a successful care management program. Commonwealth Fund Issue Brief, 19(9), 1-19.
Hornik, K., Stinchcombe, M., & White, H. (1989). Multilayer feedforward networks are universal approximators. Neural Networks, 2(5), 359-366.
Kim, D. H., & Schneeweiss, S. (2014). Measuring frailty using claims data for pharmacoepidemiologic studies of mortality in older adults: Evidence and recommendations. Pharmacoepidemiology and Drug Safety, 23(9), 891-901.
Liaw, W., Moore, M., Iko, C., & Bazemore, A. (2015). Lessons for primary care from the first ten years of Medicare coordinated care demonstration projects. J Am Board Fam Med, 28(5), 556-564.
McCarthy, D., Ryan, J., & Klein, S. (2015). Models of care for high-need, high-cost patients: An evidence synthesis. Commonwealth Fund Issue Brief, 31, 1-19.
Mauguen, A., & Begg, C. B. (2016). Using the Lorenz curve to characterize risk predictiveness and etiologic heterogeneity. Epidemiology, 27(4), 531.
Nichol, K. L., Nordin, J., Mullooly, J., Lask, R., Fillbrandt, K., & Iwane, M. (2003). Influenza vaccination and reduction in hospitalizations for cardiac disease and stroke among the elderly. New England Journal of Medicine, 348(14), 1322-1332.
Peikes, D., Chen, A., Schore, J., & Brown, R. (2009). Effects of care coordination on hospitalization, quality of care, and health care expenditures among Medicare beneficiaries: 15 randomized trials. JAMA, 301(6), 603-618.
Pelser, C., Henderson, M., & Stockwell, I. (2019, Apr. 23). Risk factors for potentially avoidable hospital admissions and emergency department visits: A literature review. Baltimore, Md.: The Hilltop Institute, UMBC.
Ruggles, S., Flood, S., Goeken, R., Grover, J., Meyer, E., Pacas, J., & Sobek, M. (2019). IPUMS USA: Version 9.0 [data set]. Minneapolis, Minn.: IPUMS. https://doi.org/10.18128/D010.V9.0
Steyerberg, E. W., Vickers, A. J., Cook, N. R., Gerds, T., Gonen, M., Obuchowski, N., Pencina, M. J., & Kattan, M. W. (2010). Assessing the performance of prediction models: A framework for some traditional and novel measures. Epidemiology, 21(1), 128.
Tu, J. V. (1996). Advantages and disadvantages of using artificial neural networks versus logistic regression for predicting medical outcomes. Journal of Clinical Epidemiology, 49(11), 1225-1231.
U.S. Census Bureau. (2018a). TIGER/Line® Shapefiles. Technical documentation. Retrieved from https://www2.census.gov/geo/pdfs/maps-data/data/tiger/tgrshp2018/TGRSHP2018_TechDoc.pdf
U.S. Census Bureau. (2018b). ZIP Code Tabulation Areas (ZCTAs). Retrieved from https://www.census.gov/programs-surveys/geography/guidance/geo-areas/zctas.html
U.S. Postal Service Office of Inspector General. (2013). The untold story of the ZIP code. Report numbers RARC-WP-13-006. Retrieved from https://www.uspsoig.gov/sites/default/files/document-library-files/2015/rarc-wp-13-006_0.pdf
Walter, S., & Tiemeier, H. (2009). Variable selection: Current practice in epidemiological studies. European Journal of Epidemiology, 24(12), 733.

What is claimed is:

1. A method comprising:
receiving, from a database, data associated with a plurality of individuals;
correlating the data to an avoidable healthcare event for one or more of the individuals;
generating a model that relates the data to the avoidable healthcare event based on the correlation of the data to the avoidable healthcare event;
applying the model to data of another individual to generate an indicator of risk of the other individual to the avoidable healthcare event; and
presenting the indicator of risk via a user interface.

2. The method of claim 1, wherein the generated model is a regression model.

3. The method of claim 1, wherein the data associated with a plurality of individuals comprises one or more of diagnoses of the individuals, healthcare procedures of the individuals, medications of the individuals, utilization of healthcare services, demographic information, and geographic locations associated with the individuals.

4. The method of claim 1, wherein the data in the avoidable healthcare event comprises one of hospitalization and emergency facility visit.

5. The method of claim 1, wherein the applying the model comprises applying the model to data of the other individual to generate an indicator of risk of the other individual to the avoidable healthcare event within a predetermined period of time.

6. The method of claim 1, wherein the indicator of risk is a score.

7. The method of claim 1, wherein the steps of receiving, correlating, generating and apply are implemented at a first computing device comprising at least one processor and memory, and
wherein the step of presenting is implemented at a second computing device comprising the user interface.

8. The method of claim 7, further comprising communicating, by the first computing device, the indicator to the second computing device.

9. The method of claim 1, wherein the steps of receiving, correlating, generating and apply are implemented at a first computing device comprising at least one processor and memory, and
wherein receiving the data comprises receiving the data from a second computing device comprising memory including the database.

10. A system comprising:
a database comprising data associated with a plurality of individuals;
an adverse healthcare event analyzer configured to:
correlate the data to an avoidable healthcare event for one or more of the individuals;
generate a model that relates the data to the avoidable healthcare event based on the correlation of the data to the avoidable healthcare event; and
apply the model to data of another individual to generate an indicator of risk of the other individual to the avoidable healthcare event; and
a user interface configured to present the indicator of risk.

11. The system of claim 10, wherein the generated model is a regression model.

12. The system of claim 10, wherein the data associated with a plurality of individuals comprises one or more of diagnoses of the individuals, healthcare procedures of the individuals, medications of the individuals, utilization of healthcare services, demographic information, and geographic locations associated with the individuals.

13. The system of claim 10, wherein the data in the avoidable healthcare event comprises one of hospitalization and emergency facility visit.

14. The system of claim 10, wherein the adverse healthcare event analyzer is configured to apply the model to data of the other individual to generate an indicator of risk of the other individual to the avoidable healthcare event within a predetermined period of time.

15. The system of claim 10, wherein the indicator of risk is a score.

16. The system of claim 10, wherein the functions of receiving, correlating, generating and apply are implemented at a first computing device comprising at least one processor and memory, and
wherein the function of presenting is implemented at a second computing device comprising the user interface.

17. The system of claim 16, wherein the first computing device is configured to communicate the indicator to the second computing device.

18. The system of claim 10, wherein the functions of receiving, correlating, generating and apply are implemented at a first computing device comprising at least one processor and memory, and
wherein the data is received at the first computing device from a second computing device comprising memory including the database.

19. A computer program product comprising a computer readable storage medium having program instructions embodied therewith, the program instructions executable by a computing device to cause the computing device to:
receive, at the computing device, data associated with a plurality of individuals;
correlate, at the computing device, the data to an avoidable healthcare event for one or more of the individuals;
generate, at the computing device, a model that relates the data to the avoidable healthcare event based on the correlation of the data to the avoidable healthcare event;
apply, at the computing device, the model to data of another individual to generate an indicator of risk of the other individual to the avoidable healthcare event; and
present the indicator of risk via a user interface.
